(19) United States
(12) Patent Application Publication
(10) Pub. No.: US 2016/0213353 A1
(43) Pub. Date: Jul. 28, 2016

(54) ULTRASOUND IMAGING APPARATUS, ULTRASOUND IMAGING METHOD AND ULTRASOUND IMAGING PROGRAM

(71) Applicants: Hironari MASUI, Hamamatsu (JP); Takashi AZUMA, Tokyo (JP)

(72) Inventors: Hironari MASUI, Hamamatsu (JP); Takashi AZUMA, Tokyo (JP)

(21) Appl. No.: 14/241,536

(22) PCT Filed: Jul. 27, 2012
(86) PCT No.: PCT/JP2012/069244

§ 371 Date: Dec. 30, 2014

(30) Foreign Application Priority Data
Oct. 28, 2011 (JP) 2011-237670

(51) Int. Cl. A61B 8/08; A61B 8/00; A61B 8/14

(52) U.S. Cl. CPC A61B 8/5207; A61B 8/14; A61B 8/469; A61B 8/5223; A61B 8/463; A61B 8/5246

(57) ABSTRACT

The present invention is to provide an ultrasound imaging apparatus which generates a scalar field image directly without obtaining a motion vector, so as to make tissue boundaries discernible.

A received signal is processed to generate images of two or more frames, two frames are selected from the images, multiple regions of interest are set in one frame, and in the other frame, search regions each wider than the region of interest are set respectively for the multiple regions of interest. In the search region, multiple candidate regions each in a size corresponding to the region of interest are provided. Then, a norm is obtained between a pixel value of the region of interest and a pixel value of the candidate region, for each of the multiple candidate regions, thereby obtaining a norm distribution within the search region. An image is generated assuming a value representing the state of the norm distribution as a pixel value of the region of interest that is associated with the search region.

### TECHNICAL FIELD

The present invention is directed to a technique that relates to an ultrasound imaging method and an ultrasound imaging apparatus, allowing a tissue boundary to be clearly discerned, upon imaging a living body through the use of ultrasound waves.

### BACKGROUND ART

In an ultrasound imaging apparatus used for medical diagnostic imaging, there is known a method for estimating a distribution of elastic modulus of tissues, based on an amount of change in a small area of a diagnostic image sequence (B-mode image), converts a degree of stiffness to a color map for display. However, in the case of peripheral zone of tumor, for instance, there is not found a large difference in acoustic impedance nor in elastic modulus, relative to surrounding tissue, and in this situation, it is not possible to discern the boundary between the tumor and the surrounding tissue, in the diagnostic image sequence nor in the elasticity image.

Therefore, there is a method for obtaining a motion vector of each region in a diagnostic image, according to a block matching process on two diagnostic image data items being different chronologically, and generating a scalar field image from the motion vectors. With this configuration, it is possible to discern the tissue boundaries where neither the acoustic impedance nor the elastic modulus is different significantly from the surroundings.

However, in a region of the image data including high noise, such as a marginal domain for signal penetration where echo signals become faint, an error vector may occur due to noise influence, upon obtaining the motion vector, and this may deteriorate the discerning degree of the boundary. Therefore, in the Patent Document 1, upon obtaining the motion vector, a degree of similarity of image data is calculated between a region of interest and multiple regions as destination candidates of the region of interest, and according to a distribution of the degree of similarity, a degree of reliability is determined as to the motion vector that is obtained with regard to the region of interest. If the degree of reliability is low, it is possible to remove the motion vector, or the like, and therefore this may enhance the degree of discerning the boundary.

### PRIOR ART DOCUMENT

### Patent Document

### Patent Document 1

PCT International Publication No. WO2011/052602

### DISCLOSURE OF THE INVENTION

### Problem to be Solved by the Invention

The method for discerning the tissue boundary by obtaining the motion vector, as described in the Patent Document 1, and the like, needs two steps; firstly, obtaining the motion vector of each region on the image by the block matching process, and secondly, converting the motion vector into scalar to generate a scalar field image.

An object of the present invention is to provide an ultrasound imaging apparatus which generates the scalar field image directly without obtaining the motion vector, so as to make the boundaries in a test subject discernible.

### Means to Solve the Problem

In order to achieve the object above, according to a first aspect of the present invention, the ultrasound imaging apparatus as described in the following will be provided. In other words, the ultrasound imaging apparatus incorporates a transmitter configured to transmit an ultrasound wave to an object, a receiver configured to receive an ultrasound wave coming from the object, and a processor 10, configured to process the received signal in the receiver and generate images of at least two frames. The processor sets multiple regions of interest in one frame, out of at least the two frames of images being generated, and sets on one of the other frames, search regions each wider than the region of interest, respectively for the multiple regions of interest. The processor sets in the search region, multiple candidate regions each having a corresponding size of the region of interest, and obtains norm between a pixel value of the region of interest and a pixel value of the candidate region, for each of the multiple candidate regions, thereby obtaining a norm distribution within the search region and generating a value (scalar value) representing a state of the norm distribution, as a pixel value of the region of interest that is associated with the search region.

### Effect of the Invention

According to the present invention, a value representing the state of the norm distribution in the search region is obtained. If there is a boundary, the norm indicates a low value along the boundary. With the configuration above, an image is generated assuming the value representing the state of the norm distribution (scalar value), as the pixel value of the region of interest being associated with the search region, and therefore, it is possible to generate an image showing the boundaries of a test subject, without generating a vector field.

### BRIEF DESCRIPTION OF DRAWINGS

FIG. 1 is a block diagram illustrating a system configuration example of the ultrasound imaging apparatus according to the first embodiment;

FIG. 2 is a flowchart illustrating a processing procedure for generating an image by the ultrasound imaging apparatus according to the first embodiment;

FIG. 3 is a flowchart illustrating details of the step 24 in FIG. 2;

FIG. 4 illustrates the process of the step 24 in FIG. 2 through the use of a test subject (phantom) having a double-layered structure;

FIG. 5(a) illustrates a distribution chart indicating the p-norm distribution in the search region, when the region of interest is in a static part; FIG. 5(b) illustrates a histogram of the p-norm distribution as shown in FIG. 5(a); FIG. 5(c) illustrates a distribution chart indicating the p-norm distribution in the search region, when the region of interest is in the boundary part according to the first embodiment; and FIG. 5(d) illustrates a histogram of the p-norm distribution as shown in FIG. 5(c);

FIG. 6(a) illustrates a B-mode image of the first embodiment, FIG. 6(b) illustrates a scalar field image of the first embodiment, FIG. 6(c) illustrates a conventional vector field image, and FIG. 6(d) illustrates a strain tensor image of the conventional vector field image;

FIG. 7(a) illustrates a superimposed image of the scalar field image and the B-mode image, according to the first embodiment, FIG. 7(b) illustrates a superimposed image of the scalar field image, the B-mode image, and the vector field image, according to the first embodiment;

FIG. 8 is a flowchart showing a processing procedure for image generation by the ultrasound imaging apparatus according to the first embodiment;

FIG. 9(a) illustrates an image showing an example where a virtual image is generated in the scalar field image; FIG. 9(b) illustrates a histogram showing an average value and frequency of the p-norm values according to the second embodiment; and FIG. 9(c) illustrates the scalar field image in which a low-reliability portion is replaced with a dark color display according to the second embodiment;

FIG. 10 is a flowchart showing a procedure for processing an image according to the second embodiment;

FIG. 11 is a flowchart showing a procedure for generating an image according to the third embodiment;

FIG. 12(a) to FIG. 12(h) illustrate models in eight directions to be set in the search region of the third embodiment;

FIG. 13(a) to FIG. 13(c) illustrate pattern examples each showing the orientation of the boundary and the vector field according to the sixth embodiment;

FIG. 14 is a flowchart showing a procedure for obtaining a boundary norm according to the sixth embodiment;

FIG. 15 illustrates ROIs being configured by partial superimposition according to the seventh embodiment;

FIG. 16 is a flowchart showing a processing procedure for reducing computation by using a look-up table according to the seventh embodiment;

FIG. 17 is a graph showing information entropy that is obtained as to successive frames according to the eighth embodiment:

FIG. 18 is a flowchart showing a processing procedure for displaying an image that uses the information entropy according to the eighth embodiment;

FIG. 19(a) illustrates a superimposed image of the extracted scalar field image, the vector field image, and the B-mode image, according to the ninth embodiment; and FIG. 19(b) illustrates a histogram of the scalar values and frequency of the scalar field image; and

FIG. 20 is a flowchart showing a processing procedure for generating the scalar field image that is extracted according to the ninth embodiment.

### BEST MODE FOR CARRYING OUT THE INVENTION

The ultrasound imaging apparatus of the present invention is provided with a transmitter configured to transmit an ultrasound wave to an object, a receiver configured to receive the ultrasound wave coming from the object, and a processor configured to process a received signal in the receiver and generate images of at least two frames. The processor sets multiple regions of interest in one frame, out of the two or more frames of images being generated, and sets in one of the other frames, search regions each wider than the region of interest, respectively for the multiple regions of interest. In the search region, there are provided multiple candidate regions, each in a size corresponding to the region of interest. The processor obtains the norm between the pixel value of the region of interest and the pixel value of the candidate region, for each of the multiple candidate regions, thereby obtaining the norm distribution within the search region, and generating an image assuming a value representing the state of the norm distribution (scalar value) as the pixel value of the region of interest that is associated with the search region. Here, it is also possible to calculate the norm by directly using an amplitude value or a phase value of the received signal, instead of the pixel value, in the region of interest. A linear change is reflected on the original received signal more accurately and higher resolution may be achieved relative to the pixel value, since logarithmic compression processing is applied to the pixel value.

If there is a boundary, the norm indicates a low value along the boundary. Therefore, an image is generated by assuming the value representing the state of the norm distribution (scalar value) as the pixel value of the region of interest that is associated with the search region, and accordingly, the ultrasound imaging apparatus of the present invention is allowed to generate an image representing the boundary of the test subject, without generating a vector field.

As the norm, the p-norm (also referred to as “power norm”) expressed by the following formula (1) may be employed.

$$\begin{matrix}[ Formula 1 ] & \\ p \text{-} Norm = {( {\sum }_{i , j} {\vert {p}_{m} ( {i}_{0} , {j}_{0} ) - {p}_{m + Δ} ( i , j ) \vert}^{p} )}^{1 / p} & ( 1 )\end{matrix}$$

It is to be noted here that Pₘ(i₀, j₀) represents a pixel value of the pixel at a predetermined position (i₀, j₀) (e.g., a center position) within the region of interest, P_{m+Δ}(i, j) represents a pixel value of the pixel at the position (i, j) within the candidate region, and p represents a real number being predetermined.

It is desirable that the aforementioned p is a real number being larger than 1.

As the value representing the state of the norm distribution (scalar value), statistics of the norm distribution may be employed. For example, as the statistics, it is possible to use a rate of divergence that is defined by a difference between a minimum norm value and an average value of the norm values in the norm distribution within the search region. It is alternatively possible to use a coefficient of variation as the statistics, which is obtained by dividing a standard deviation of the norm values by the average value, in the norm distribution within the search region.

As the value representing the state of the norm distribution (scalar value), a value other than the statistics may be used. By way of example, a first direction and a second direction are obtained, out of multiple direction centering on a specific region being set within the search region; the first direction in which the average of the norm values in the candidate regions located along the direction, becomes a minimum and the second direction passing through the specific region and being orthogonal to the first direction. Then, it is possible to use a value of ratio or a value of difference between the average of the norm values in the candidate regions along the first direction and the average of the norm values in the candidate regions along the second direction, as the value representing the state of the norm distribution as to the region of the interest that is associated with the search region. On this occasion, the norm distribution within the search region may be subjected to enhancement in advance, using the Laplacian filter, and the value of ratio or the value of difference may be obtained as to the distribution after the enhancement.

Alternatively, a matrix may be generated representing the norm distribution within the search region, an eigenvalue decomposition process is applied to the matrix to obtain an eigenvalue, and then this eigenvalue may be used as the value (scalar value) representing the state of the norm distribution as to the region of interest that is associated with the search region.

It is also possible to configure such that the processor further obtains the motion vector. By way of example, the processor selects as a destination of the region of interest, the candidate region in which the norm value becomes minimum in the search region, and obtains the motion vector that connects the position of the region of interest and the position of the candidate region being selected. The motion vector is generated for each of the multiple regions of interest, thereby generating the motion vector field. It is further possible for the processor to obtain as a boundary norm value, a total sum of a squared value of derivative of y direction with respect to x component and a squared value of derivative of x direction with respect to y component, as to each of multiple specific regions set in the motion vector field, and generates an image assuming the boundary norm value as the pixel value of the specific region.

If multiple regions of interest are set in a partially overlapping manner, it is possible to configure such that the processor stores a value obtained with regard to the overlapping region in a lookup table of the storage region, upon calculating the norm as to one region of interest, and the processor reads the value from the lookup table and uses the value, upon calculating the norm as to another region of interest. Similarly, if multiple candidate regions are set in a partially overlapping manner, it is also possible to store a value obtained with regard to the overlapping region in the lookup table of the storage region, and the processor reads the value from the lookup table and uses the value, upon calculating the norm as to another candidate region. Those configurations above may reduce the computations.

It is further possible for the processor to generate multiple frames of images on a time-series basis, the image being generated assuming the value representing the state of the norm distribution as the pixel value, calculates the amount of information entropy for each frame. If the amount of information entropy is smaller than a predetermined threshold, it may be determined not use the image as the image for displaying the frame. This configuration allows elimination of an abnormal image with a small amount of information entropy, enabling display of successive images with preferable visibility.

It is also possible to generate an extraction image that is obtained by extracting pixels each having a value representing the norm distribution, the value being equal to or larger than a predetermined value, and display the extraction image in a superimposed manner on the B-mode image. Since the pixel with the value representing the norm distribution, being equal to or higher than the predetermined value, corresponds to a pixel indicating a boundary, the extraction image may be displayed only on the boundary part in the B-mode image. In order to define the predetermined value, a histogram may be generated as to the value representing the state of the norm distribution and its frequency, with regard to the image that is generated assuming the value representing the state of the norm distribution as the pixel value. The histogram is searched for a bell-shaped distribution, and a minimum value of the bell-shaped distribution may be used as the aforementioned predetermined value.

The ultrasound imaging apparatus according to another aspect of the present invention is provided with a transmitter configured to transmit an ultrasound wave to an object, a receiver configured to receive the ultrasound wave coming from the object, and a processor configured to process a received signal in the receiver and generate images of at least two frames. The processor sets multiple regions of interest in a distribution of the received signals that correspond to one frame, out of the received signals corresponding to two or more frames of images being received. The processor sets a search region wider than the region of interest in another one frame, for each of the multiple regions of interest. The processor sets within the search region, multiple candidate regions in a size corresponding to the region of interest. The processor obtains the norm, between an amplitude distribution or a phase distribution in the region of interest, and an amplitude distribution or a phase distribution in the candidate region, for each of the multiple candidate regions, thereby obtaining the norm distribution within the search region. The processor generates an image assuming the value representing the state of the norm distribution, as the pixel value of the region of interest that is associated with the search region.

In addition, according to the present invention, an ultrasound imaging method is provided. In other words, the method transmits an ultrasound wave to an object, processes a received signal obtained by receiving the ultrasound wave coming from the object, and generates images of at least two frames. The method selects two frames from the images, and sets multiple regions of interest in one frame. The method sets a search region wider than the region of interest in the other frame, for each of the multiple regions of interest. The method sets multiple candidate regions each in a size corresponding to the region of interest within the search region. The method obtains the norm between the pixel value in the region of interest and the pixel value in the candidate region, for each of the multiple candidate regions, thereby obtaining the norm distribution within the search region. Then, the method generates an image, assuming the value representing the state of the norm distribution, as the pixel value of the region of Interest that is associated with the search region.

According to the present invention, a program for imaging ultrasound waves is provided. In other words, this program is provided for ultrasound imaging, allowing a computer to execute a first step to fifth step. In the first step, two frames are selected from ultrasound images of at least two frames. In the second step, multiple regions of interest is set in one frame. In the third step, a search region is set wider than the region of interest in another one frame for each of the multiple regions of interest. And multiple candidate regions are set in a size corresponding to the region of interest within the search region. In the fourth step, the norm between the pixel value in the region of interest and the pixel value in the candidate region is obtained, for each of the multiple candidate regions, thereby a norm distribution is obtained within the search region. In a fifth step, an image is generated assuming the value representing the state of the norm distribution as the pixel value of the region of interest that is associated with the search region.

A specific explanation will be provided as to the ultrasound imaging apparatus according to one embodiment of the present invention.

### First Embodiment

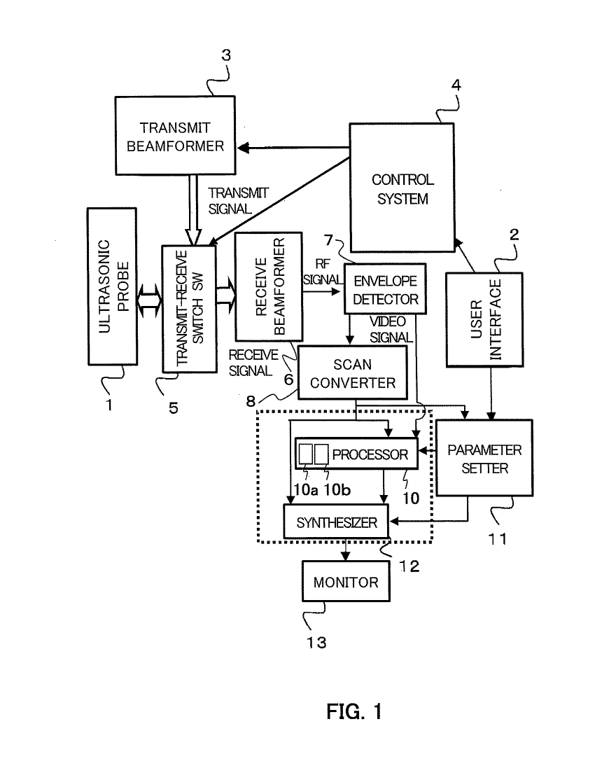

FIG. 1 illustrates a system configuration of the ultrasound imaging apparatus according to the present embodiment. This apparatus is provided with a ultrasound boundary detecting function. As illustrated in FIG. 1, this apparatus is provided with an ultrasound probe (probe) 1, a user interface 2, a transmit beamformer 3, a control system 4, a transmit-receive switch 5, a receive beamformer 6, an envelope detector 7, a scan converter 8, a processor 10, a parameter setter 11, a synthesizer 12, and a monitor 13.

The ultrasound probe 1 on which the ultrasound elements are provided in one-dimensional array, serves as a transmitter configured to transmit an ultrasound beam (an ultrasound pulse) to a living body. The ultrasound probe 1 serves as a receiver configured to receive an echo signal (a received signal) reflected from the living body. Under the control of the control system 4, the transmit beamformer 3 outputs a transmit signal having a delay time in accordance with a transmit focal point. And the transmit signal is sent to the ultrasound probe 1 via the transmit-receive switch 5. The ultrasound beam is reflected or scattered within the living body and returned to the ultrasound probe 1. The ultrasound beam is converted to electrical signals by the ultrasound probe 1, and transferred to the receive beamformer 6 as the received signal, via the transmit-receive switch 5.

The receive beamformer 6 is a complex beam former for mixing two received signals which are out of phase by 90 degrees. The receive beamformer 6 performs a dynamic focusing to adjust the delay time in accordance with a receive timing under the control of the control system 4, so as to output radio frequency signals corresponding to the real part and the imaginary part. The envelope detector 7 detects the radio frequency signals. The signals are converted into video signals. The video signals are inputted into the scan converter 8, so as to be converted into image data (B-mode image data). The configuration described above is the same as the configuration of a well-known ultrasonic imaging apparatus. Further in the present invention, it is possible to implement ultrasound boundary detection according to the configuration for directly processing the RF signal.

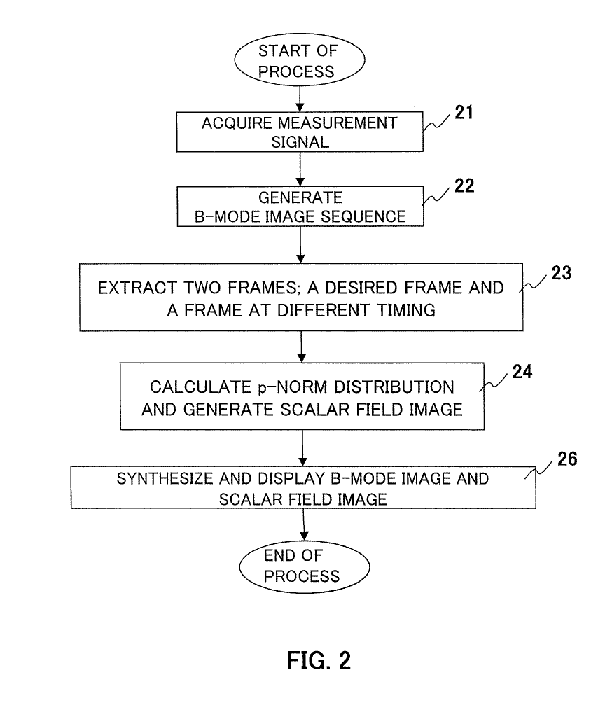

In the apparatus of the present invention, the processor 10 implements the ultrasound boundary detection process. The processor 10 incorporates a CPU 10a and a memory 10b. The CPU 10a executes the program stored in the memory 10b in advance, thereby generating a scalar field image on which tissue boundaries in the test subject are detectable. With reference to FIG. 2, and the like, a process for generating the scalar field image will be explained in detail later. The synthesizer 12 performs processing for synthesizing the scalar field image and the B-mode image, and then displays the combined image on the monitor 13.

The parameter setter 11 performs a setting of parameters for the signal processing in the processor 10, and a setting for selecting an image for display in the synthesizer 12. An operator (a device operator) inputs those parameters from the user interface 2. As for the parameters for the signal processing, for instance, it is possible to accept from the operator a setting of the region of interest on a desired frame m, and a setting of a search region on the frame m+A that is different from the frame m. As for the setting for selecting the image for display, for instance, it is possible to accept from the operator a setting for selecting either one of the following to be displayed on a monitor; one image being obtained by synthesizing an original image and a vector field image (or a scalar image), and a sequence of at least two images being placed side by side.

FIG. 2 is a flowchart that shows an operation of processing for generating an image and synthesizing images in the processor 10 and the synthesizer 12 according to the present invention. The processor 10 firstly acquires a measurement signal from the scan converter 8, subjects the measurement signal to an ordinal signal processing to generate a B-mode image sequence (steps 21 and 22). Next, the processor extracts two frames from the B-mode image sequence, a desired frame m and a frame m+Δ at a different timing (step 23). By way of example, it is assumed that Δ=1 frame, and two frames, that is, the desired frame m and the next frame m+1 are extracted. It is possible to configure this extraction of two frames as being accepted from the operator via the parameter setter 11. It is also possible that two frames are selected by the processor 10 automatically.

The processor 10 calculates a p-norm distribution from thus extracted two frames, and generates a scalar field image (step 24). The processor generates a synthesized image obtained by superimposing the scalar field image being generated on the B-mode image, and display the synthesized image on the monitor 13 (step 27). It is further possible that in the step 23, frames sequentially different on a time-series basis are selected as the desired frame, the aforementioned steps 21 to 27 are repeated. The synthesized images are successively displayed, thereby displaying a moving picture made up of the synthesized images.

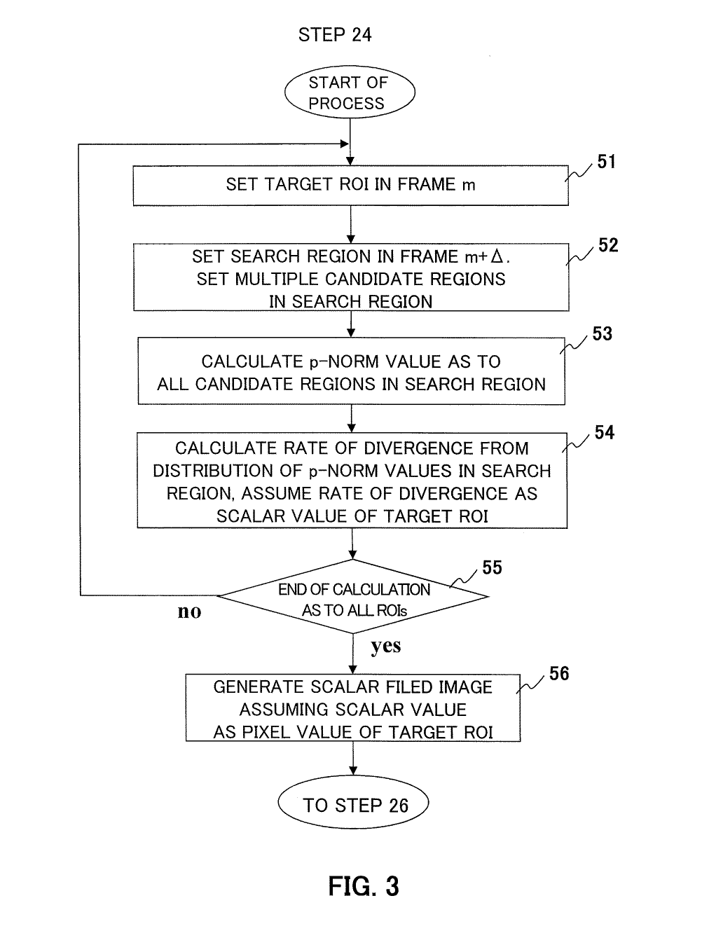

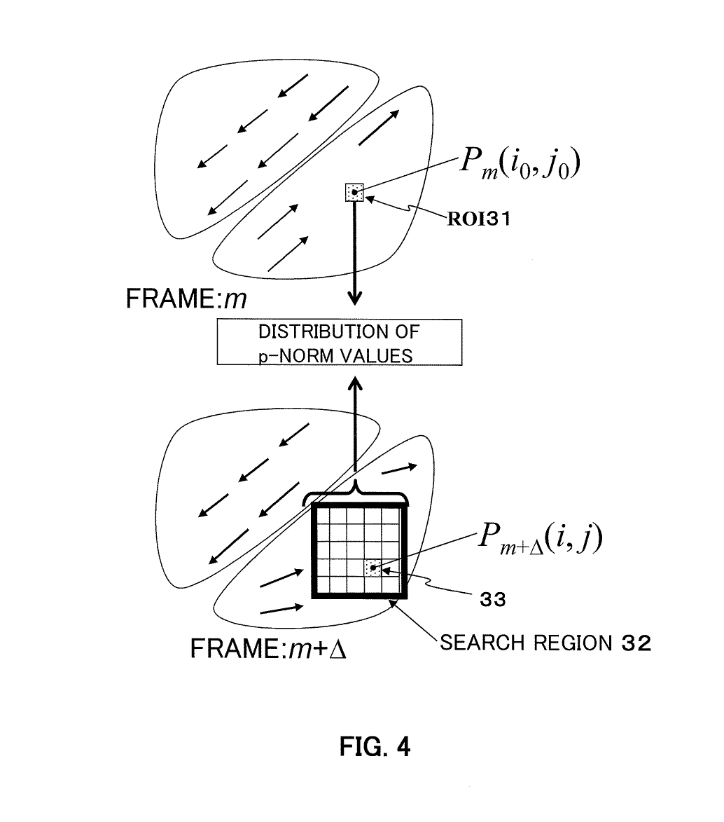

FIG. 3 is a flowchart showing a detailed process of the operation for generating the scalar field image of the aforementioned step 24. Firstly, the processor 10 sets an ROI (region of interest) 31 in the frame m extracted in the step 23, as shown in FIG. 4, a ROI includes a predetermined number N of pixel (step 51). A value of the pixel included in the ROI 31, which may be a brightness distribution for instance, is represented as Pₘ(i₀, j₀). Here, the item “i₀, j₀” indicates a position of the pixel within the ROI 31.

Next, as shown in FIG. 4, the processor 10 sets the search region 32 in a predetermined size, within the frame m+Δ that is extracted in the step 23 (step 52). The search region 32 includes the position of the ROI 31 in the frame m. By way of example, the search region 32 is configured as matching the center position of the ROI 31. The size of the search region 32 is set to be a predetermined size that is larger than the ROI 31. Here, an explanation will be provided as to the configuration where the ROI 31 is sequentially set on all over the image of the frame m, and the search region 32 is provided in a certain size centering on each ROI 31. However, it is also possible to set the ROI 31 sequentially only within a predetermined range of the frame m, the range being accepted from the operator in the parameter setter 11.

The processor 10 sets multiple candidate regions 33 within the search region 32, each candidate region having the size being equal to the size of the ROI 31 as shown in FIG. 4. In FIG. 4, the search region 32 is partitioned like a matrix into the size being equal to the ROI 31, thereby setting the candidate regions 33. It is further possible to provide neighboring candidate regions 33 in such a manner as partially overlapping. The value of the pixel included in the candidate region 33, being the brightness distribution, for instance, is represented as P_{m+Δ}(i, j). Here, the item “i, j” indicates a position of the pixel within the candidate region 33.

The processor 10 uses the brightness distribution P_{m+Δ}(i, j) of the pixels in the candidate region 33 and the brightness distribution Pₘ(i₀, j₀) in the ROI 31 to calculate p-norm according to the aforementioned formula (1), and sets this p-norm as the p-norm value of the candidate region 33. By the aforementioned formula (1), P-th power of the absolute value of the difference is calculated between the brightness Pₘ(i₀, j₀) of the pixel at the position (i₀, j₀) in the ROI 31, and the brightness P_{m+Δ}(i, j) of the pixel at the position (i, j) in the candidate region 33 being associated with the position (i₀, j₀). Then by the formula (1), the value of the P-th power is added up, as to all the pixels in the candidate region 33, and raised to the 1/p-th power, and this result is the p-norm obtained. As the p-value, a predetermined real value, or a value accepted from the operator via the parameter setter 11 may be employed. The p-value is not limited to an integer, but it may be a decimal number.

The p-norm including “p” as power, as shown in the aforementioned formula (1), is a value corresponding to a concept of distance, and the p-norm represents similarity between the brightness distribution Pₘ(i₀, j₀) in the ROI 31 and the brightness distribution P_{m+Δ}(i, j) in the candidate region 33. In other words, if the brightness distribution Pₘ (i₀, j₀) in the ROI 31 is identical to the brightness distribution P_{m+Δ}(i, j) in the candidate region 33, the p-norm becomes zero. The larger is the difference between both the brightness distributions, the larger becomes the value.

The processor 10 calculates the p-norm value, as to all the candidate regions 33 in the search region (step 53). Accordingly, it is possible to obtain the p-norm distribution within the search region 32 that is associated with the ROI 31. The p-norm value thus obtained is stored in the memory 10b in the processor 10.

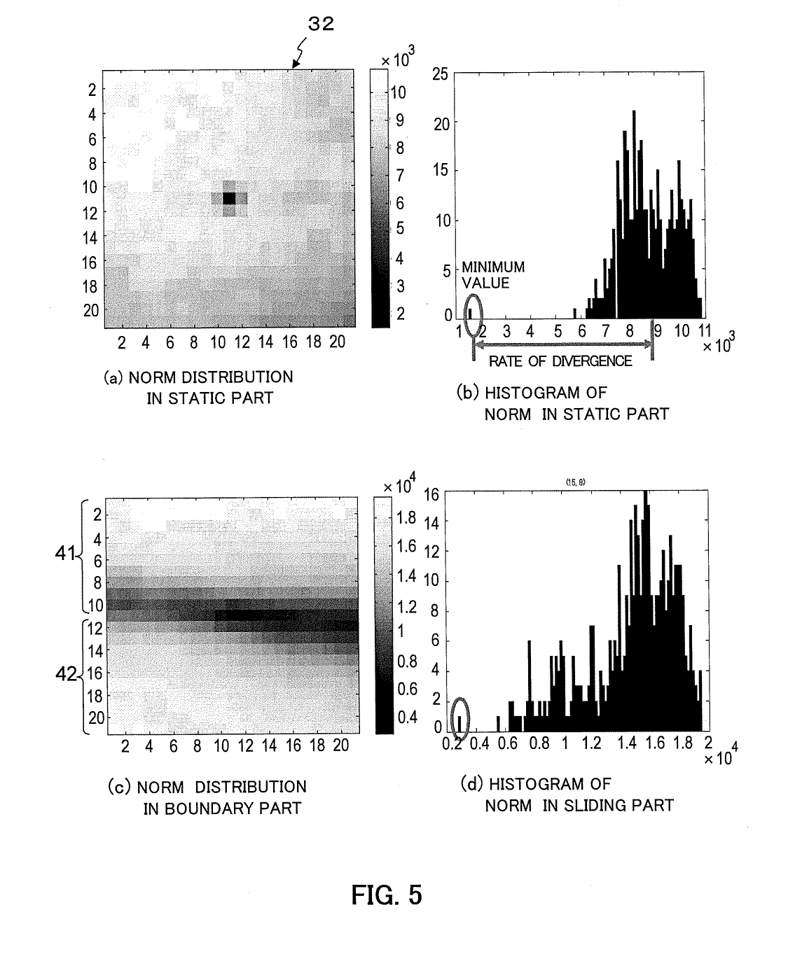

FIG. 5(a) and FIG. 5(c) illustrate examples of the p-norm distribution according to the present invention. FIG. 5(a) illustrates the norm distribution in the search region 32, in the case where both the ROI 31 and the search region 32 thereof are positioned at a static part of the test subject. FIG. 5(c) illustrates the norm distribution in the search region 32, in the case where the phantom 41 and the phantom 42 being made of a gel material serving as a test subject, are superimposed one on another, and the ROI 31 is placed on the boundary where the lower-side phantom slides relatively in horizontal direction with respect to the upper-side phantom 41. It is to be noted that in FIG. 5(a) and FIG. 5(c), the search region 32 is partitioned into 21×21 candidate regions 33. The candidate region 33 is 30×30 pixels and the search region 32 is 50×50 pixels, in block size. Then, the candidate region 33 is made to shift, pixel by pixel within the search region 32. In other words, 29 pixels overlap between the neighboring candidate regions 33. The center of the search region 32 corresponds to the position of the ROI 31.

As shown in FIG. 5(a), if the ROI 31 is positioned in the static part, the center position corresponding to the position of the ROI 31 indicates a minimum norm value in the p-norm distribution. On the other hand, as shown in FIG. 5(c), if the ROI 31 is placed on the boundary of the test subject, being sliding, not only the center position of the search region 32 becomes the minimum norm value, but also there is formed an area where the p-norm value is small (p-norm valley) in the norm distribution, in the direction along the boundary of the test subject within the search region 32.

As described above, the p-norm distribution is different depending on whether the ROI 31 is positioned in the static part of the test subject, or in the boundary being sliding, and the present invention utilizes this difference to create an image. Specifically, the statistics indicating the p-norm distribution in the search region 32 is obtained, and the obtained statistics is assumed as a scalar value of the ROI 31 that is associated with this search region (step 54). Any statistics may be applicable, as far as it is able to represent a difference of the p-norm distribution between the static part and the boundary part. Here, a rate of divergence obtained in the formula (2) is used as the statistics:

[Formula 2]

Rate of Divergence≡S̅−Sₘᵢₙ (2)
S̅: Average of p-norm values
Sₘᵢₙ: Minimum p-norm value
   In other words, the processor 10 obtains the minimum value and the average value from the entire p-norm values in the search region 32, and calculates the rate of divergence according to the formula (2).

Histograms in FIG. 5(b) and FIG. 5(d) illustrate the rate of divergence according to the formula (2). FIG. 5(b) and FIG. 5(d) are histograms indicating the p-norm values within the search region 32, as illustrated in FIG. 5(a) and FIG. 5(c), respectively. As shown in FIG. 5(a), if the ROI 31 is positioned in the static part, the p-norm distribution of the search region 32 indicates a minimum norm value at the center position corresponding to the position of the ROI 31, and p-norm values surrounding the center are high. Therefore, there is found the characteristics that sufficiently wide divergence exists between the minimum value of the p-norm values and the average of the distribution thereof. Therefore, the rate of divergence becomes high. On the other hand, as shown in FIG. 5(c), in the case where the ROI 31 is positioned in the boundary part, the p-norm value at the center position corresponding to the ROI 31 becomes the minimum value, but in the surrounding area thereof, the p-norm values also become small by an error. The distribution of overall histogram is spreading. Therefore, a difference between the minimum value of the p-norm and the average of the distribution thereof becomes smaller, thereby reducing the rate of divergence.

As discussed above, in the step 54, the rate of divergence of the p-norm distribution (scalar value) is obtained. According to the scalar value, it is possible to indicate whether the ROI 31 is positioned in the static part or in the sliding part of the test subject.

The aforementioned steps 51 to 54 are repeated until the calculation is carried out as to all the ROIs 31 (step 55). The rates of divergence (scalar values) obtained as to all the ROIs 31 are converted into image pixel values (e.g., brightness values), and thereby generating an image (scalar field image) (step 56). According to the steps 51 to 56 as described above, the scalar field image of the step 24 is generated.

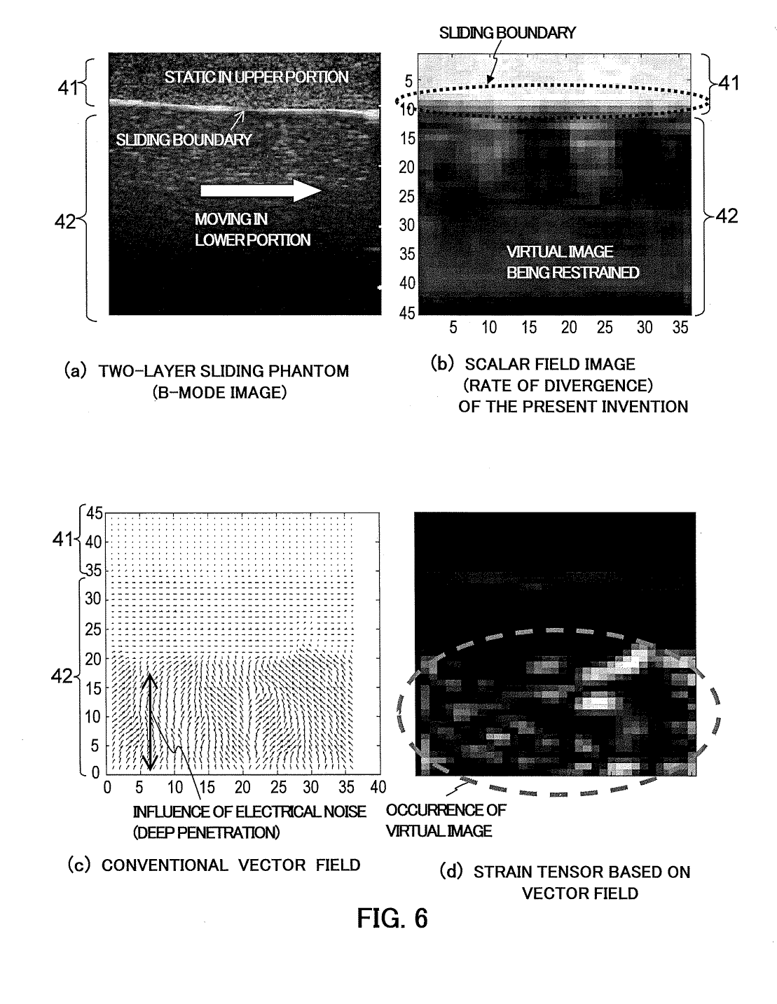

FIG. 6(a) illustrates the B-mode image obtained in the step 22, and FIG. 6(b) illustrates a specific example of the scalar field image obtained in the step 24. The B-mode image in FIG. 6(a) is obtained by superimposing the gel-material phantoms 41 and 42 one on another in two-layers, and performing the imaging along with lateral movement of the upper phantom 41 on which the ultrasound probe us fixed. The upper phantom 41 on which the ultrasound probe is fixed, is relatively in the static state. On the other hand, the lower phantom 42 is in the vector field indicating lateral movement. It is to be noted that calculations are carried out assuming p=2 in FIG. 6(b).

As shown in FIG. 6(b), it is found that the scalar field image of the present invention, which uses as the pixel value, the rate of divergence (scalar value) in the p-norm distribution of the present invention, shows a high rate of divergence in the sliding boundary part between the phantoms 41 and 42. A clear image of the sliding boundary is successfully generated. Therefore, in the step 26, the scalar field image is displayed independently or in a superimposed manner on the B-mode image, thereby allowing the boundary that is hardly represented in the B-mode image to be displayed clearly by the scalar field image; for example, for the case where neither the acoustic impedance nor the elastic modulus is significantly different from the surroundings.

Further in the scalar field image as shown in FIG. 6(b), no virtual image is generated even in the deep area around the marginal domain of signal penetration, and it is found that the virtual image is successfully restrained.

As a comparative example, FIG. 6(c) shows a vector field image obtained by a conventional block matching process. This vector field image is obtained by assuming the B-mode image in FIG. 6(a) as the frame m, obtaining a moved position of the ROI according to the block matching process (step 24) with the next frame (frame m+Δ, Δ=1 frame), and indicating the direction and the magnitude of the movement by a vector (arrow). In FIG. 6(c), there is found in the lower part (deep part) a phenomenon that the motion vectors are in the state of turbulent. As the distance becomes larger from the probe installed on the upper portion, the S/N ratio of detection sensitivity is lowered, resulting in significant influence by the electrical noise, and the like. This causes the situation above, indicating the limitations of signal penetration. FIG. 6(d) illustrates an image generated by obtaining strain tensor based on the vector field as shown in FIG. 6(c) and using the strain tensor as the pixel value (e.g., brightness value). In FIG. 6(c) similarly, there is found in the deep part, a phenomenon that virtual image is generated, due to the influence of the error vectors.

As discussed above, in the tensor field image (FIG. 6(b)) obtained from the p-norm according to the present embodiment, it is possible to restrain the virtual image and show the sliding boundary more clearly, in comparison to the conventional vector field and the strain tensor field image based on the conventional vector field as shown in FIG. 6(c) and FIG. 6(d).

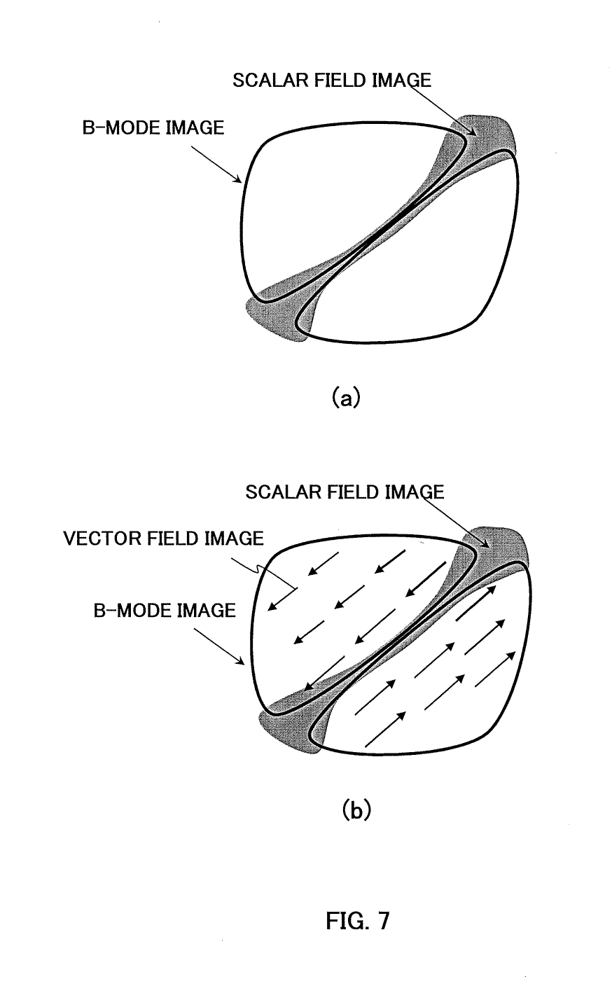

The scalar field image and the B-mode image obtained in the present invention are displayed in a manner superimposing one on another as shown in FIG. 7(a). Accordingly, even in the case where the boundary in the B-mode image is unclear, the scalar field image allows the boundary to be discerned.

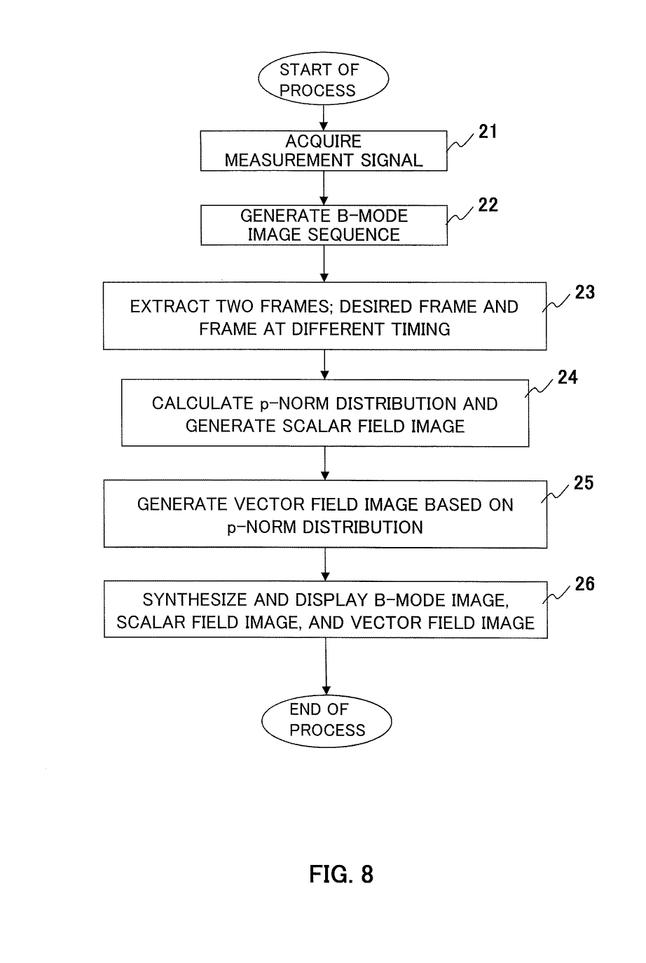

In the present embodiment, it is further possible to generate the vector field image, and display this vector field image, the scalar field image, and the B-mode image in a superimposed manner. In this case, the process in step 25 is performed after the step 24, as shown in FIG. 8. The process in the step 25 searches the p-norm values being calculated in the step 24 for a minimum value, in all of the candidate regions 33 within the search region 32, determines the candidate region 33 having the minimum value as a destination region of the ROI 31. The process in the step 25 decides a motion vector connecting the position (i₀, j₀) of the ROI 31, with the position (iₘᵢₙ, jₘᵢₙ) of the destination candidate region. This process in the step 25 is executed for all the ROIs 31, thereby obtaining the vector field. An image showing each vector in the form of arrow is generated, and the vector field image is obtained.

The vector field image being obtained, the scalar field image, and the B-mode image are displayed in a superimposed manner (step 26). FIG. 7(b) illustrates an example of the superimposed images. By displaying the vector field image in a superimposed manner, it is possible to ascertain clearly in which direction the test subject moves, placing therebetween the boundary that is discerned in the scalar field image.

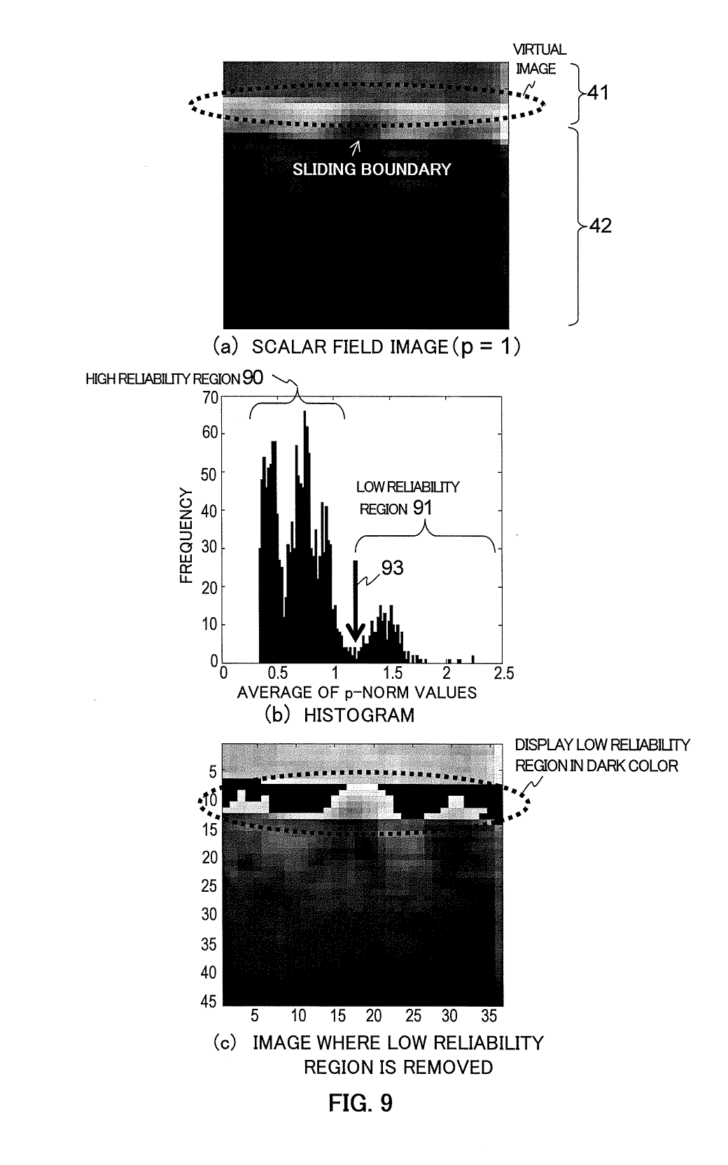

In the present embodiment, it is sufficient if the p-value of the p-norm in the formula (1) is a real number. However, a parameter survey may be conducted on the p-value using appropriate variation width, with respect to a typical sample of the evaluation target, for instance. An optimum p-value may be set as a value which enables acquisition of a clear image with the least virtual images. In addition, it is desirable that the p-value is a real number larger than 1. FIG. 9(a) illustrates an image example in the case where the setting is p=1, that is a scalar field image obtained according to the flows as shown in FIG. 2 and FIG. 3, with regard to the B-mode image of the two-layer sliding phantom of FIG. 6(a). In FIG. 9(a) it is found that in the image lower part (deep part), a virtual image is restrained in a similar manner as the case of p=2, as shown in FIG. 6(b). However, in the static region upper than the boundary, there is found a new virtual image. This result leads us to surmise that the norm with p=1 is vulnerable to influence of sparseness. Accordingly, in order to restrain the virtual image in the static part, it is desirable that p is set to be a value larger than 1. Here, it is also possible to perform image processing for eliminating the virtual image. As for this matter, an explanation will be provided in the second embodiment.

In the explanation above, the rate of divergence is obtained as the statistics representing the distribution of the p-norm in the search region 32, and the scalar field image is generated based on this value, but it is also possible to use a parameter other than the rate of divergence. By way of example, it is possible to use a coefficient of variation. The coefficient of variation is defined by the following formula. It is statistics obtained by normalizing the standard deviation by the average, representing the magnitude of variation in the distribution (i.e., a degree of difficulty in minimum value separation).

$$\begin{matrix}[ Formula 3 ] & \\ Coefficient of Variation ≡ \frac{{σ}_{S}}{\overline{S}} \\ {σ}_{S} \text{:} Standard Deviation \\ \overline{S} \text{:} Average of p \text{-} norm values & ( 3 )\end{matrix}$$

### Second Embodiment

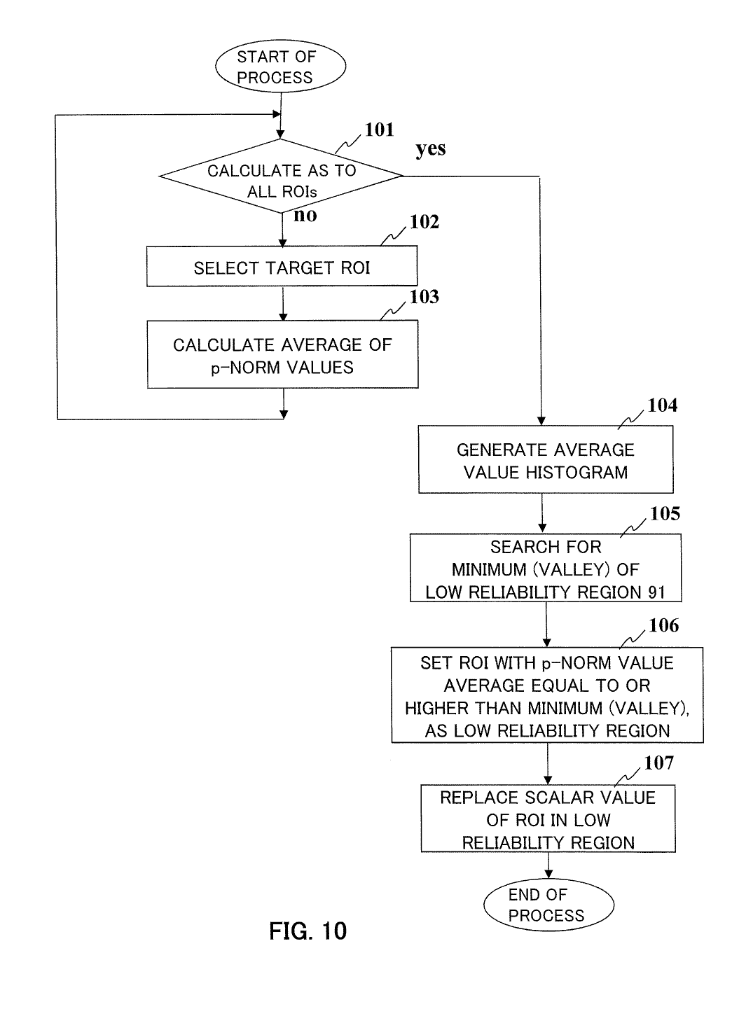

In the second embodiment, if any virtual image occurs in the scalar field image obtained in the first embodiment, this virtual image may be removed, or the like. In other words, a degree of reliability of the image region is identified, and a region with a low reliability is removed, or the like, thereby eliminating the virtual image and enhancing the reliability of the entire image. This will be explained with reference to FIG. 9 and FIG. 10.

FIG. 9(a) illustrates the scalar field image obtained assuming p=1 in the formula (1), as described in the first embodiment, and the virtual image is generated in the boundary. FIG. 9(b) illustrates a histogram that is used for identifying the degree of reliability, and FIG. 9(c) illustrates a scalar field image in which the brightness in the low-reliability region is replaced by a dark color. FIG. 10 is a flowchart showing the operation of the processor 10 for removing the virtual image.

Upon receiving an instruction for removing the virtual image from the operator, the processor 10 reads and executes a program for removing the virtual image, and operates as shown in the flow of FIG. 10. Firstly, one of the multiple ROIs 31 set in the step 51 in the flow of FIG. 3 is selected (step 102). A p-norm value obtained as to each of the multiple candidate regions 33 within the search region 32 associated with the ROI 31 is read from the memory 10b in the processor 10. An average of those values is calculated to obtain the average value of the p-norm values corresponding to the ROI 31 (step 103). Those steps 102 and 103 are repeated as to all the ROIs 31 (step 101).

A histogram as shown in FIG. 9(b) is generated from the average value and frequency of the p-norm values being obtained as to all the ROIs 31 (step 104). It is estimated that the larger is the average value of the p-norm values, the lower is the degree of reliability of the ROI. If there is a bell-shaped distribution with a low peak in the histogram, within a range where the average value of the p-norm values is large, this range is determined as a low reliability region 91. In other words, the bell-shaped distribution with a high peak, positioned in the range where the average value of the p-norm values is small, is determined as a high reliability region 90. The bell-shaped distribution with a low peak positioned in the range where the average value of the p-norm values is larger, is determined as the low reliability region 91. Then, a position of the valley (minimum frequency value) 93 between the low reliability region 91 and the high reliability region 90 is obtained (step 105). The ROI 31 in the range where the average value of the p-norm values is larger than the valley 93 (low reliability region 91) is determined as the low-reliability region (step 106). In terms of this ROI 31, the scalar value (the rate of divergence or the coefficient of variation) as obtained in the step 54 in FIG. 3 is eliminated, and then, the scalar field image is generated (step 107). By way of example, as shown in FIG. 9(c), the ROI 31 of the low reliability region is displayed, being replaced by a predetermined dark color to which certain brightness is assigned in advance. It is further possible to display the ROI 31, replacing the brightness of the ROI 31 in the low reliability region with a predetermined light color, or with the same brightness as the surroundings.

Since the second embodiment enables elimination of the virtual image, it is possible to provide a scalar field image on which the boundary of the test subject can be discerned more clearly.

### Third Embodiment

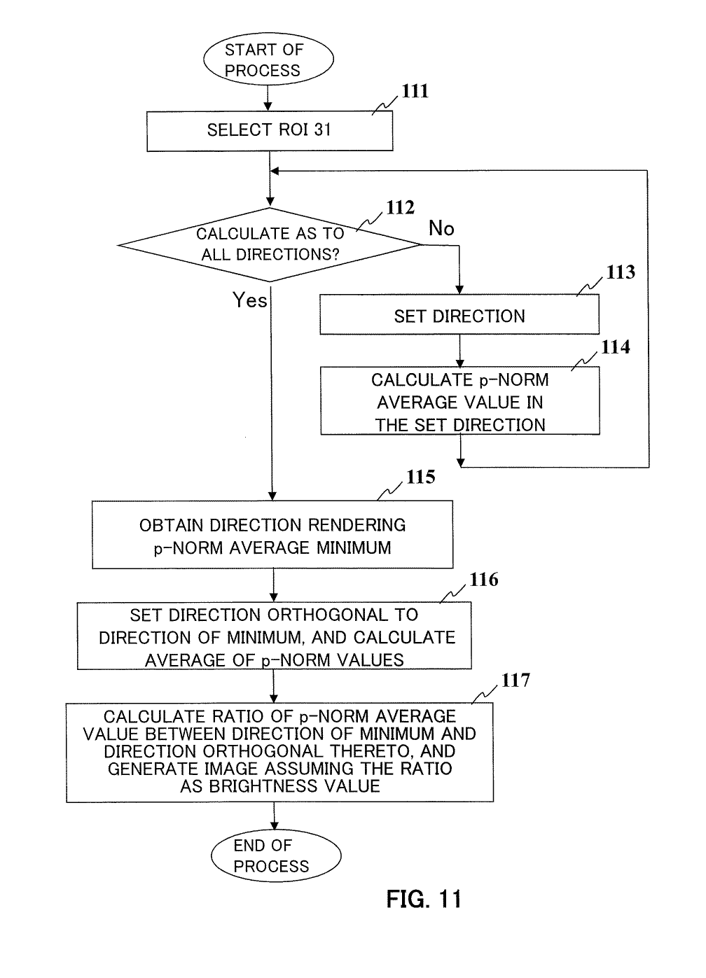

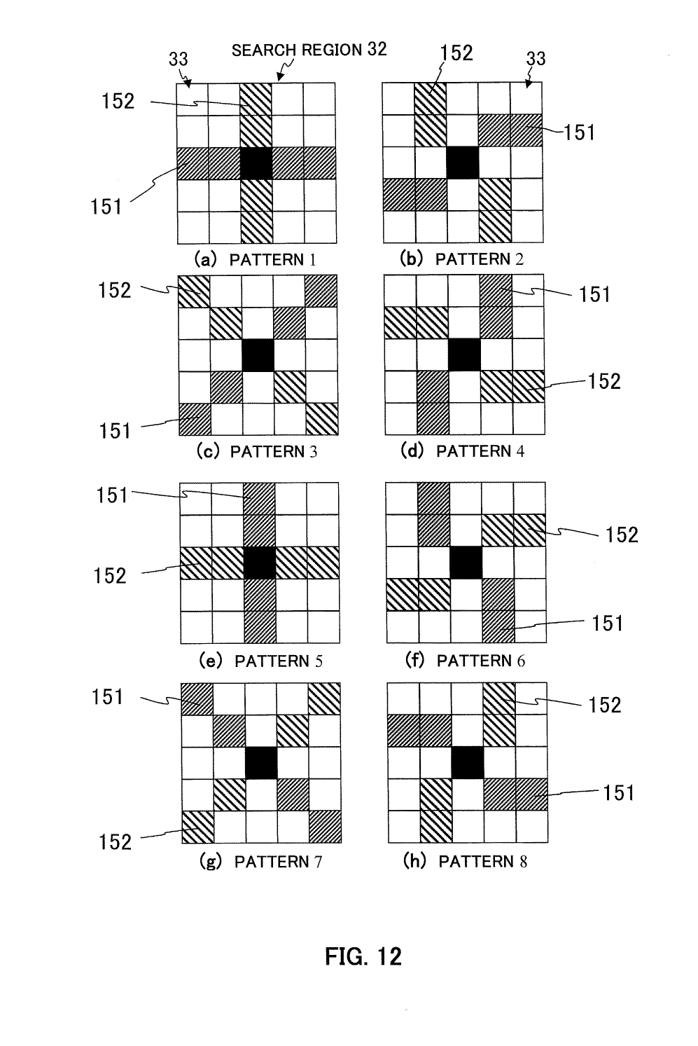

In the first embodiment, statistics (the rate of divergence or the coefficient of variation) of the p-norm distribution is obtained to generate an image. In the third embodiment, an image is generated from the p-norm distribution where a tissue boundary is discernible, through the use of a different method. This processing method will be explained with reference to FIG. 11 and FIG. 12.

In the p-norm value distribution in the search region 32 as described in the first embodiment, the candidate region 33 along the boundary in the test subject forms a region with small p-norm values (a valley of p-norm values) along the boundary. Therefore, the distribution of p-norm values has the characteristics that the values of the candidate region 33 along the boundary indicate smaller values than the candidate region 33 in the direction orthogonal to the boundary. With the use of the characteristics, an image is generated in the present embodiment.

FIG. 11 illustrates a processing flow of the processor 10 according to the present embodiment. In addition, FIG. 12(a) to FIG. 12(h) illustrate eight patterns of the candidate regions 33 being selectable on the p-norm value distribution of the search region 32. It is to be noted that in FIG. 12(a) to FIG. 12(h), for the ease of illustration, the candidate regions 33 are arranged within the search region 32 in the 5×5 matrix-like form, for instance, but actual arrangement of the candidate regions 33 corresponds to the arrangement as set in the step 52.

Firstly, the processor 10 executes the processing from the step 21 to the step 23 in FIG. 2 and from the step 51 to the step 53 in FIG. 3 according to the first embodiment. Accordingly, a p-norm value distribution with regard to the search regions 32 in association with the multiple ROIs 31 is obtained. Next, the ROI 31 is selected (step 111). A predetermined direction 151 passing through the center of the search region 32 is set as shown in FIG. 12(a), in the norm value distribution of the search region 32 that is associated with the ROI 31. By way of example, in the case of FIG. 12(a), the predetermined direction is a horizontal direction. Multiple candidate regions 33 are selected being positioned along the set direction 151, and an average of the norm values of those candidate regions 33 is obtained (step 114).

The processes of the steps 113 and 114 are performed as to each of the eight directions 151 respectively illustrated in the eight patterns as shown in FIG. 12(a) to FIG. 12(h) (step 112). In the pattern of FIG. 12(b), the predetermined direction 151 is a direction inclined counterclockwise approximately at 30° with respect to the horizontal direction. In the patterns of FIG. 12(c) to FIG. 12(h), the predetermined direction 151 is inclined counterclockwise with respect to the horizontal direction, approximately at 45°, 60°, 90°, 120°, 135°, and 150°, respectively.

A direction 151 in which the average of the p-norm values becomes a minimum value is selected out of the eight predetermined directions 151 (step 115). Next, the direction 152 orthogonal to the selected direction 151 is provided, and an average of the p-norm values of the candidate regions 33 being positioned along the direction 152 is obtained (step 116). The directions 152 orthogonal to the eight directions 151 are as illustrated in FIG. 12(a) to FIG. 12(h), respectively. A ratio (=the average of p-norm values in the orthogonal direction 152/the average of the p-norm values in the minimum direction 151) is calculated, between the average of the p-norm values in the direction 151 where the average value of the p-norm values becomes a minimum, being selected in the step 115, and the average of the p-norm values in the direction 152 orthogonal to the selected direction 151 being obtained in the step 116. This ratio is assumed as the pixel value (e.g., the brightness value) of the target ROI 31. By executing those processing above as to all the ROIs 31, thereby generating an image (step 117).

Since the candidate regions along the boundary of the search region 32 in the test subject have small p-norm values (valley), the ratio obtained in the step 117 becomes a larger value, compared to the ROI 31 that is not located on the boundary. Therefore, by assuming the ratio as the pixel value, it is possible to generate an image which allows clear discerning of the boundary.

In the present embodiment, the ratio of the average of the p-norm values is used, but this is not the only example. It is also possible to employ other function values, such as a different value between the average of the p-norm values in the minimum direction 151 and the average of the p-norm values in the orthogonal direction 152.

In the explanation above, as shown in FIG. 12(a) to FIG. 12(h), the present invention is directed to a configuration to obtain a boundary in the test subject from the valley of the p-norm value distribution in the candidate regions 33, each arranged within the search region 32. However, the present invention is not limited to this example, but it is further possible to obtain the boundary in the test subject from the distribution of the pixel values within one candidate region 33, according to a similar method. Specifically, it is considered that in FIG. 12(a) to FIG. 12(h), the search region 32 is replaced by the candidate regions 33, and the candidate regions 33 within the search region 32 are replaced by pixels. For this case, in the example of FIG. 12(a) to FIG. 12(h), one candidate region 33 is configured by 5×5 pixels. In the candidate region 33, eight directions 151 passing through the central pixel of the candidate region 33, and the directions 152 respectively orthogonal thereto are provided. Each of the eight directions 152 respectively orthogonal to the eight directions 151, is made up of five pixels. If the pixel value of the five pixels in each of the directions is assumed as P_{m+Δ}(i, j). The pixel value of the central pixel of the five pixels is assumed as Pₘ(i₀, j₀). The p-norm value of the five pixels in the direction is calculated according to the formula of the first embodiment. Thus obtained p-norm value is divided by the number of pixels (5, in the case of five pixels), thereby calculating the p-norm average value. This p-norm average value is obtained for each of the eight directions 151 as shown in FIG. 12(a) and FIG. 12(b). The direction 151 where the p-norm average value becomes a minimum is selected. A ratio of the p-norm average value between the direction 151 having the minimum value, and the direction 152 orthogonal thereto is calculated.

In the candidate region 33 positioned at the boundary part of the test subject, the p-norm average value of the pixels in the direction along the boundary (the direction 151 along which the p-norm average value becomes minimum) is small, and the p-norm average value in the direction 152 being orthogonal thereto is large. Therefore, the ratio therebetween becomes a large value. On the other hand, in the candidate region 33 positioned in a homogeneous area other than the boundary of the test subject, the p-norm average value in the direction 151 and the p-norm average value in the direction 152 being orthogonal thereto becomes equivalent. Therefore the ratio therebetween becomes nearly 1. When the ratio is calculated as to the candidate regions 33 of the entire image of a target frame, the pixels in the candidate region 33 with a large ratio may correspond to the boundary part. Thus by generating an image assuming the ratio, as the pixel value of the central pixel of the candidate region 33, it is possible to generate an image which allows estimation of the boundary in units of pixels. Instead of the ratio, it is further possible to use another function value such as a difference value between the p-norm average value in the direction 151 having the minimum value and the p-norm average value in the direction 152 orthogonal thereto.

### Fourth Embodiment

The fourth embodiment will be explained.

In the fourth embodiment, prior to subjecting the p-norm distribution in the search region 32 to the processing of FIG. 11 by the processor 10 in the third embodiment, Laplacian filter is applied to the p-norm distribution, and the p-norm distribution is subjected to enhancement. By subjecting the p-norm distribution to the enhancement, the valley of the p-norm value along the boundary direction is enhanced. Thereafter, the processing of the third embodiment as shown in FIG. 11 is performed, and this enables acquisition of an image that has a significant contrast in the obtained ratio, and the like, between the boundary and the region other than the boundary.

Specifically, the processes in the steps 21 to 23 in FIG. 2 and in the steps 51 to 53 in FIG. 3 according to the first embodiment are executed, and a distribution of the p-norm values is obtained as to the search regions 32 respectively associated with the multiple ROIs 31. The spatial second-derivative image processing (Laplacian filter) is applied to thus obtained distribution of p-norm values to generate the p-norm value distribution in which the outline of the valley of the p-norm values along the boundary direction is enhanced. The p-norm value distribution after the Laplacian filter is applied is subjected to the processing of FIG. 11 according to the third embodiment, and an image is generated.

Similarly, when the boundary of the test subject is obtained from the distribution of the pixel values within the candidate region 33 as explained in the latter half of the third embodiment, it is also possible that the Laplacian filter is applied to the pixel value distribution, subjecting the distribution to the enhancement. Thereafter, the p-norm average value or the ratio is obtained.

### Fifth Embodiment

As the fifth embodiment, an explanation will be provided as to a processing method to generate an image in which the tissue boundary is discernible from the p-norm distribution by using an eigenvalue decomposition process.

Firstly, the processor 10 executes the processes in the steps 21 to 23 in FIG. 2 and in the steps 51 to 53 in FIG. 3, and then obtains the distribution of the p-norm values as to the search regions 32 respectively associated with multiple ROIs 31. The matrix A is generated by using the p-norm values (Nₘₙ) of the candidate regions 33 within the search region 32 being obtained. The matrix A is substituted into the eigen equation as shown in the following formula (4) and eigenvalues λₙ, Δₙ₋₁, . . . and λ₁ are obtained. A maximum eigenvalue among the eigenvalues, or a linear combination of the eigenvalues is assumed as the scalar value of the ROI 31 that is associated with the search region 32. Here, the linear combination of the eigenvalues may indicate, for example, that two values, the maximum eigenvalue Δₙ and the second large eigenvalue λₙ₋₁ are used, and the function thereof, for example, (λₙ−λₙ₋₁) is assumed as the scalar value.

$$\begin{matrix}[ Formula 4 ] & \\ Av = {λ}_{i} v \\ ( i = 1 , … , n ) \\ A = [ \begin{matrix}{N}_{11} & … & {N}_{m 1} \\ ⋮ & ⋮ & ⋮ \\ {N}_{1 n} & … & {N}_{mn}\end{matrix} ] \\ {λ}_{i} = [ {λ}_{n} , {λ}_{n - 1} , … {λ}_{1} ] & ( 4 )\end{matrix}$$

Here, “Nₘₙ” represents the p-norm value obtained by the formula (1) as to the candidate regions 33 within the search region 32, and “m” and “n” indicate the positions of the candidate regions 33 within the search region 32.

The maximum eigenvalue or the linear combination of eigenvalues is obtained as the scalar value, as to all the ROIs 31, and a scalar field image is generated, assuming the scalar value as the pixel value (brightness value, or the like), similar to the step 56 in FIG. 3.

As described above, according to the present invention, the scalar field image is generated by using the eigenvalue.

In the present embodiment, the maximum eigenvalue among the eigenvalues, or the linear combination of eigenvalues is employed, but it is not limited to those examples. It is further possible to use another one or more eigenvalues.

### Sixth Embodiment

As the sixth embodiment, an explanation will be provided as to a method for generating a scalar field image that is capable of extracting a boundary based on a vector field, when a motion vector field image is generated by performing the process of the step 25 in FIG. 8 according to the first embodiment.

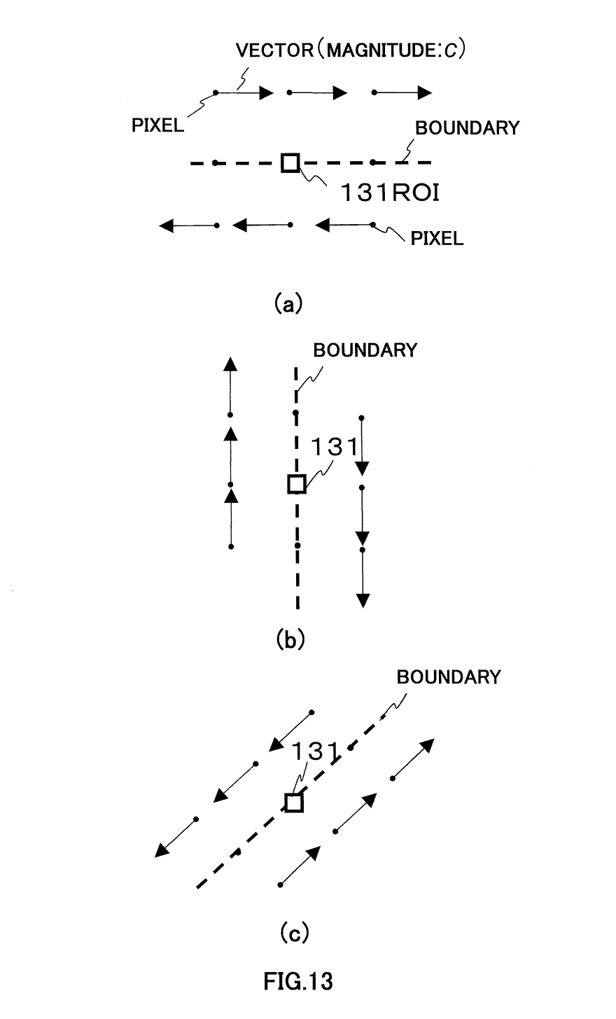

It is assumed that the motion vector field obtained in the step 25 of FIG. 8 is any of the models as shown in FIG. 13(a), FIG. 13(b), and FIG. 13 (c). The model as shown in FIG. 13(a) is an example that the direction of the boundary in the test subject is a horizontal, passing through the central ROI (specific pixel) 131 The model as shown in FIG. 13(b) is an example that the direction of the boundary is vertical. The model as shown in FIG. 13(c) is an example that the direction of the boundary is in a slanting direction at an angle of 45 degrees. It is also assumed that in the test subject, the regions placing the boundary therebetween move in the directions opposite to each other, respectively, by the motion vector with the magnitude c.

Firstly, an explanation will be provided as to the case where a conventional strain tensor is obtained for the vector field in each of those models as described above, and the strain tensor is converted into a scalar field. The formula for obtaining the strain tensor is publicly known as described in the Patent Document 2, and it is defined by the following formula:

$$\begin{matrix}[ Formula 5 ] & \\ Strain Tensor ≡ \frac{1}{2} ( \frac{\partial Y}{\partial x} + \frac{\partial X}{\partial y} ) & ( 5 )\end{matrix}$$

In the formula (5), the x-component of the motion vector is assumed as X, and the y-component thereof is assumed as Y.

The partial differential value expressed by the formula (5) is calculated as a difference average of each of the vector components on both sides of the ROI 131, for instance. Specifically, it is calculated by the formula (6) as to each of the models in FIG. 13(a), (b), and (c).

[Formula 6]

(∂Y/∂x,∂X/∂y) (6)

By way of example, in the vector field of FIG. 13(a), the result is (0, C), in the vector field of FIG. 13(b), the result is (−C, 0), and in the vector field of FIG. 13(c), the result is (−C/\sqrt{2}, C/\sqrt{2}). Therefore, even under the same condition that the vector fields on both sides placing the boundary therebetween have the magnitude C and directions opposite to each other, if the boundary is a in a slant direction as shown in FIG. 13(c), the strain tensor becomes C/2 relative to the strain tensor for the case where the boundary is in a horizontal or vertical direction as shown in FIG. 13(a) or FIG. 13(b). Therefore, if the vector field is converted into a scalar field according to the strain tensor, and an image is generated assuming the strain tensor as the pixel value of the ROI 131, the boundary in the slanting direction becomes obscure, relative to the image where the boundary is in the horizontal or vertical direction. Accordingly, the ability of detecting the boundary in the slanting direction may be impaired.

In the present invention, the motion vector field is converted into the scalar field by using the scalar value defined by the following formula (7). Since the formula (7) is in a format that includes the powers and the root of power, similar to the formula (1), it is referred to as the “boundary norm”.

$$\begin{matrix}[ Formula 7 ] & \\ Boundary Norm ≡ {( {( \frac{\partial Y}{\partial x} )}^{2} + {( \frac{\partial X}{\partial y} )}^{2} )}^{\frac{1}{2}} & ( 7 )\end{matrix}$$

When the boundary norm as shown above is obtained as to each of the models in FIG. 13(a), FIG. 13(b), and FIG. 13 (c), the boundary norm value in any of the models becomes C. Thus, irrespective of the vector direction, the vector field is allowed to be equally converted to the scalar field. Therefore, according to the boundary norm in the present invention, the vector field is converted into the scalar field, and an image is generated assuming the scalar value (boundary norm value) as the pixel value of the ROI 131. Therefore, it is possible to detect a boundary with high robustness against a directional element.

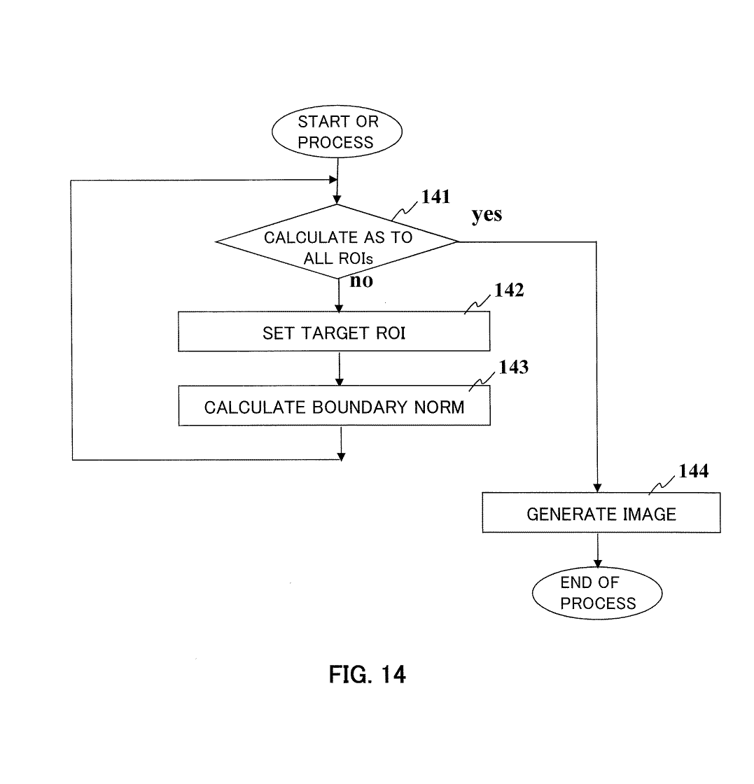

FIG. 14 illustrates a procedure for generating the scalar field image, by using the boundary norm of the present embodiment. In the beginning, the processes in the steps 21 to 25 in FIG. 8 of the first embodiment are executed, and a vector field image is generated. The processing as shown in FIG. 14 is executed on thus generated vector field image. Firstly, multiple ROIs 131 are set on the vector field image (step 142). Next, vector partial differentiation is performed in the x-direction and in the y-direction as to one ROI 131, and by using this result, the boundary norm of the formula (7) is calculated (step 143). Thus obtained boundary norm value is assumed as the scalar value of the ROI 131. Those processes above are repeated for all the ROIs 131 (step 141). Then, the boundary norm value is converted into the pixel value of the ROI 131 (e.g., brightness value), and the scalar field image is generated.

### Seventh Embodiment

The seventh embodiment will be explained.

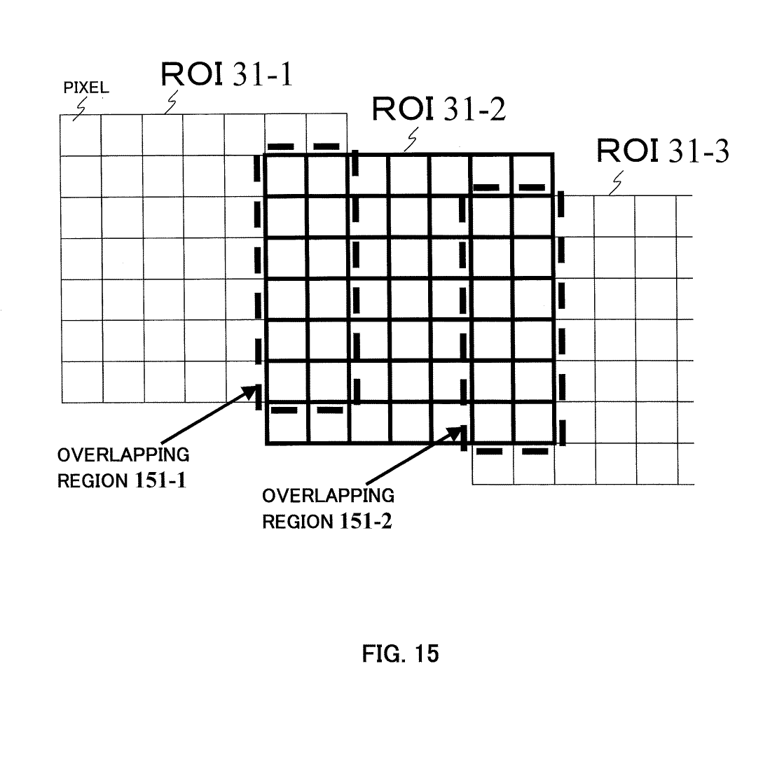

In the seventh embodiment, upon setting multiple ROIs 31 in the step 51 of the FIG. 3 according to the first embodiment, if the ROIs 31 are arranged partially overlapping as shown in FIG. 15 in order to enhance the resolution, a result of the computation in the overlapping region 151 is stored in the lookup table provided in the memory 10b of the processor 10 in the step 53, thereby reducing the amount of computation. The configuration other than the above is the same as the first embodiment.

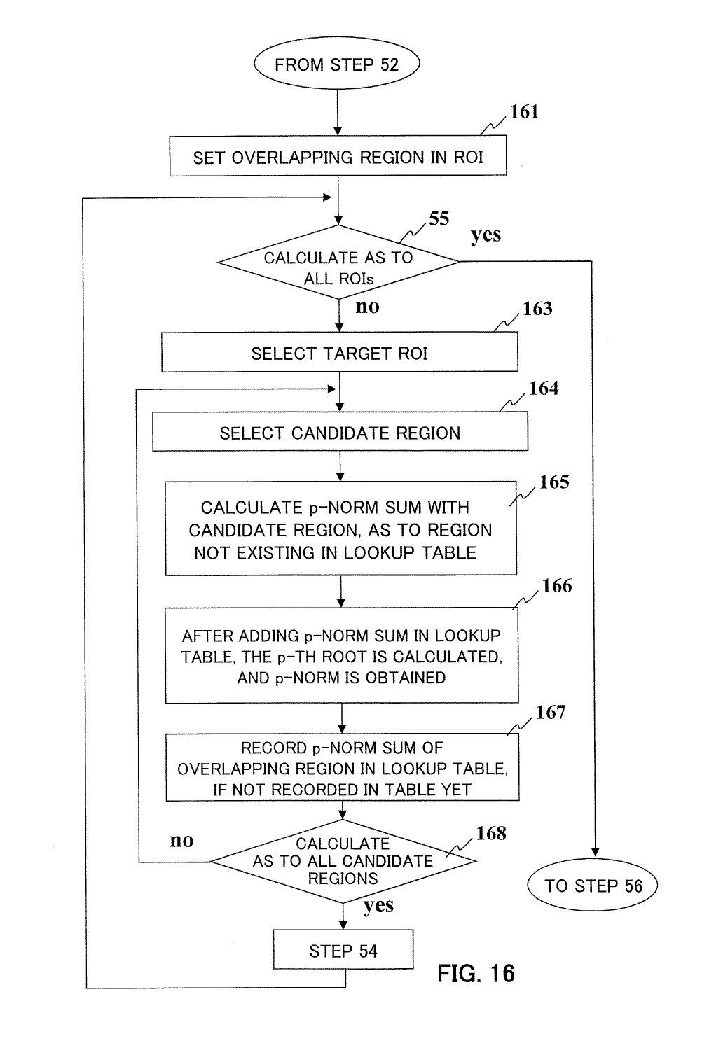

FIG. 16 illustrates a processing procedure of the step 53 according to the present embodiment. It is to be noted that in the flow of FIG. 16, the steps being the same as those in the flow of FIG. 3 are labeled the same.

Firstly, as shown in FIG. 15, in the case where overlapping regions 151-1 and 151-2 exist in the multiple ROIs 31 set in the step 51 of FIG. 2, a memory region is registered in the memory 10b of the processor 10, for recording the p-norm sum of the formula (8) described below regarding the pixels of the overlapping region, as to each candidate region 33 of the step 52, and a lookup table is generated (step 161).

Next, the target ROI 31-1 is selected (step 163), and further the candidate region 33 is selected within the search region 32 in association with the target ROI 31-1. According to the following formula (8) the p-th root of which corresponds to the formula (1), the p-norm sum is calculated, as to the pixels whose p-norm sum is not stored in the lookup table (i.e., the pixels not in the overlapping region 151-1), out of the pixels in the ROI 31-1 (step 165). It is to be noted that in the step 165, if the p-norm sum data of the overlapping region 151-1 is not recorded yet, the p-norm sum is also calculated as to the pixels in the overlapping region 151-1.

$$\begin{matrix}[ Formula 8 ] & \\ p \text{-} Norm Sum = ( {\sum }_{i , j} {\vert {p}_{m} ( {i}_{0} , {j}_{0} ) - {p}_{m + Δ} ( i , j ) \vert}^{p} ) & ( 8 )\end{matrix}$$

Next, the lookup table is referred to, and if there is stored the p-norm sum data of the pixels in the overlapping region 151-1 of the ROI 31-1, it is read out. Then, it is added to the p-norm sum obtained in the step 165, and the p-th root of the addition result is calculated, thereby obtaining the p-norm of the formula (1) (step 166). Accordingly, it is possible to obtain the p-norm value as to the candidate region of the ROI 31-1. Thus obtained p-norm value is stored in the memory 10b.

If the p-norm sum calculated in the step 166 includes the data in the overlapping region 151-1 not recorded yet in the lookup table, the p-norm sum of the overlapping region 151-1 is recorded in the lookup table (step 167). This is repeated as to all the candidate regions within the search region 32 that is associated with the ROI 31-1. Accordingly, it is possible to obtain a distribution of the p-norm values of the ROI 31-1 (step 168). After obtaining the distribution of p-norm values as to the ROI 31-1, the rate of divergence is obtained by the step 54, and it is set as the scalar value of the target ROI 31-1.

Next, the subsequent ROI 31-2 is selected (steps 162 and 163), and a candidate region is selected (step 164). According to the formula (8) the p-th root of which corresponds to the formula (1), the p-norm sum is calculated, as to a pixel whose p-norm sum is not stored in the lookup table (i.e., a pixel not in the overlapping region 151-1), out of the pixels in the ROI 31-2 (step 165). Since the p-norm sum data of the pixels in the overlapping region 151-1 of the ROI 31-2 is already stored in the lookup table, it is read out. Then, it is added to the p-norm sum obtained in the step 165, and the p-th root of the addition result is calculated, thereby obtaining the p-norm of the formula (1) (step 166). Accordingly, it is possible to obtain the p-norm value as to the candidate region of the ROI 31-2, using a small amount of computation without calculating the p-norm sum of the overlapping region 151-1.

Thus obtained p-norm value is stored in the memory 10b. The p-norm sum of the overlapping region 151-2 obtained in the calculation of the step 165 is recorded in the lookup table (step 167).

Repeating the processes in the above steps 163 to 168 as to all the ROIs allows a distribution of the p-norm values to be obtained (step 55). This eliminates the need for recalculation for the overlapping regions 151, enabling reduction of the amount of computation.

In the present embodiment, for the case where adjacent ROIs 31 are partially overlapping, an explanation has been provided as to the configuration where the overlapping region is configured and the p-norm sum thereof is stored in the lookup table. Also for the case where adjacent candidate regions 33 partially overlaps within the search region 32, an overlapping region is also configured to store the p-norm sum thereof in the lookup table, and this configuration may reduce the amount of computation.

### Eighth Embodiment

The eighth embodiment will be explained.

By executing any of the aforementioned embodiments from the first to the seventh on the successive frames, it is possible to generate a continuous image of the scalar field or a continuous image of the vector field that are obtained from the norm distribution, and display the continuous image on a time-series basis. On this occasion, there is a possibility that an abnormal frame occurs, failing to generate an appropriate image for some reason. The eighth embodiment is directed to elimination of the abnormal frame, allowing an appropriate continuous image to be displayed.

Since the abnormal frame is characterized by that a delineated area becomes extremely small, it is possible to discriminate between the abnormal frame and a normal frame according to the judgment on this point. In the present embodiment, it is determined whether the delineated area is large or small, according to the magnitude of the information entropy. The information entropy of the vector field image is defined by the following formula (9):

[Formula 9]

H=−ΣPₓ log Pₓ−ΣP_{y} log P_{y} (9)

Here, Px represents event probability of the x-component of the vector, and Py represents event probability of the y-component of the vector. The information entropy H obtained by this formula indicates the combinatory entropy of the x-component and the y-component, representing an average information amount of the entire frame.

If the information entropy is calculated as to the scalar field image that is obtained from the p-norm distribution, and the like, according to the first to the seventh embodiments, only one variable exists in the right side of the formula (9).

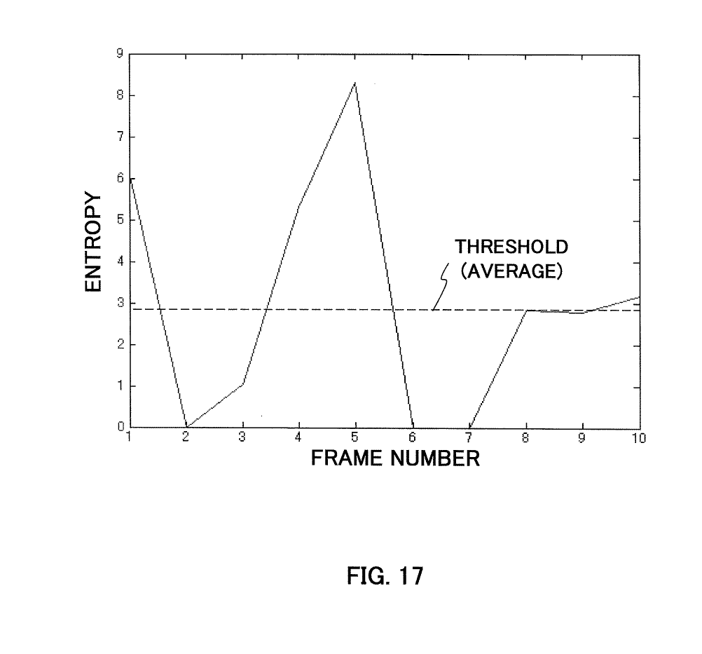

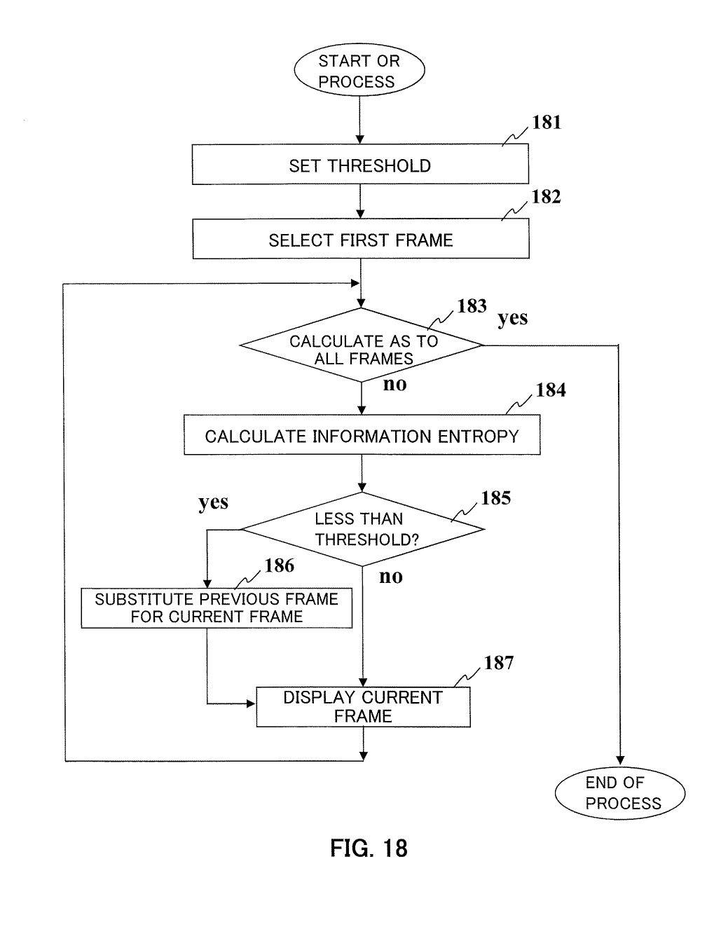

FIG. 17 illustrates a result of the information entropy that is obtained by the formula (9), with regard to one example of the successive frames on a time-series basis. FIG. 18 illustrates temporal change of the information entropy as to the successive 10 frames. FIG. 17 illustrates a processing procedure for displaying the image frames according to the present embodiment. Since the frame with small information entropy is an abnormal frame with a small amount of information, a frame with the information entropy less than a predetermined threshold is not displayed, and a previous frame is held to be displayed.

Specifically, a threshold is set by the step 181 in FIG. 18, a first frame is selected, and the information entropy is calculated. If it is less than the threshold being set, the previous frame is displayed, instead of the current frame (the present frame) as to which the information entropy is calculated, and if it is equal to or larger than the threshold, the current frame is displayed as it is. This process is repeated for all the frames. According to this process, the abnormal frame is removed, allowing the continuous image with preferable visibility to be displayed. It is to be noted that as the threshold, for example, a predetermined value may be available, or an average value obtained from a predetermined number of frames may be employed.

### Ninth Embodiment

The ninth embodiment will be explained.

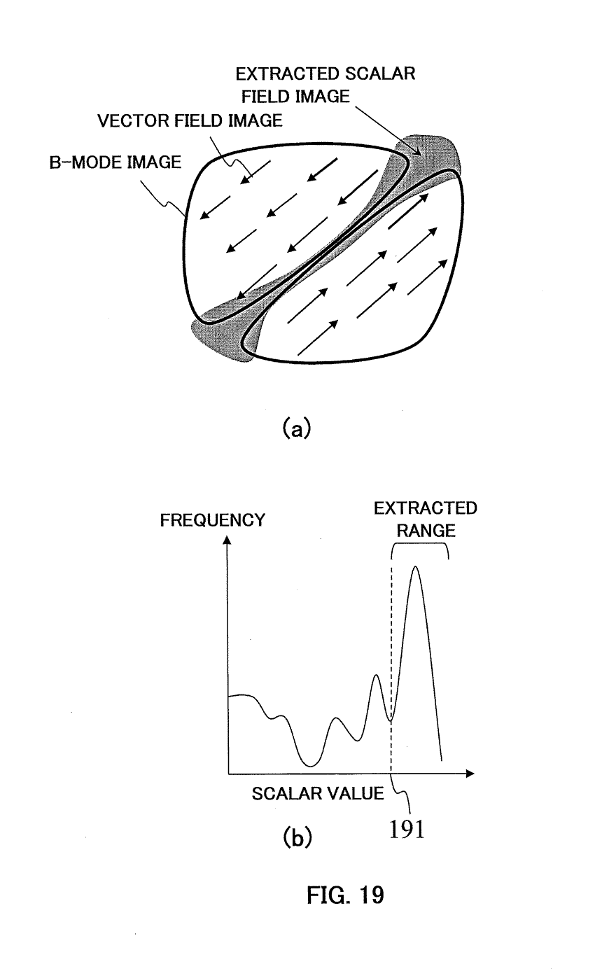

In the first embodiment, as shown in FIG. 7(a), the scalar field image obtained from the p-norm distribution and the B-mode image are displayed by superimposing one image on another, and in FIG. 7(b), those image are displayed further with a vector field image being superimposed one on another. In the ninth embodiment, only the boundary part is extracted from the scalar field image being superimposed as shown in FIG. 19(a), and thus extracted boundary part is superimposed on the B-mode image, or the like, thereby enhancing the visibility.

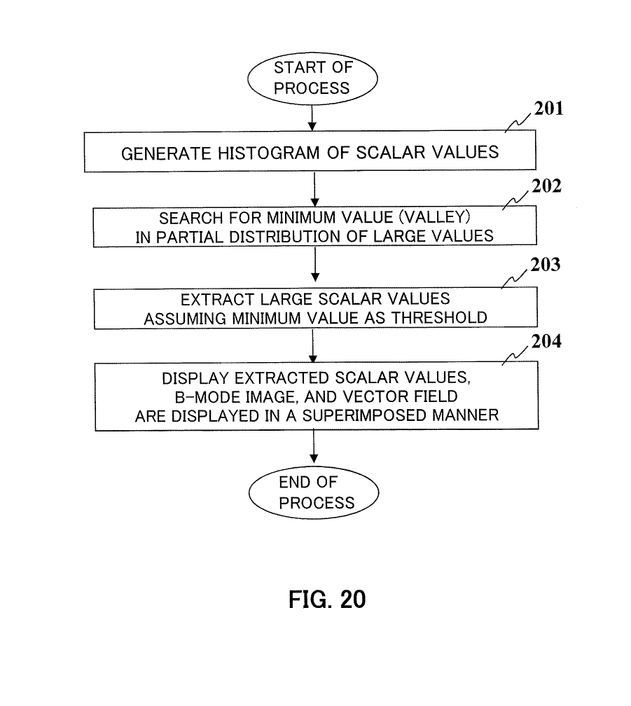

FIG. 20 illustrates a processing procedure of image synthesis according to the present embodiment. Firstly, a histogram of the scalar values of the scalar field image that is generated in the first embodiment, and the like, is made as shown in FIG. 19(b) (step 201). Searching is conducted for a bell-shaped distribution in the range where the scalar values are large, and its minimum value 191 (a valley of the distribution) is retrieved (step 202). Then, the minimum value 191 is set as the threshold, and pixels having the scalar values larger than the threshold are retrieved in the scalar field image, and an extracted scalar field image is generated (step 203). The extracted scalar field image is an image which extracts the boundary region where the scalar value is large. This extracted scalar field image is displayed in a superimposed manner on the B-mode image (and the vector field image), thereby enabling a display of an image with high visibility, where the boundary part is clearly recognizable and the region other than the boundary part is allowed to be checked easily by the B-mode image and the vector field image (step 204).

The present invention is applicable to a medical ultrasound diagnostic apparatus/treatment apparatus, and a general apparatus that uses general electromagnetic waves including ultrasound waves to measure strain and/or misalignment.

### EXPLANATION OF REFERENCES

1: ultrasound probe (probe), 2: user interface, 3: transmit beamformer, 4: control system, 5: transmit-receive switch, 6: receive beamformer, 7: envelope detector, 8: scan converter, 10: processor, 10a: CPU, 10b: memory, 11: parameter setter, 12: synthesizer, 13: monitor

What is claimed is:

1. An ultrasound imaging apparatus comprising,
a transmitter configured to transmit an ultrasound wave to an object,
a receiver configured to receive the ultrasound wave coming from the object, and
a processor configured to process a received signal in the receiver and generate images of two or more frames, wherein,
the processor
sets multiple regions of interest in one frame, out of the two or more frames of images being generated,
sets in one of the other frames, search regions each wider than the region of interest, respectively for the multiple regions of interest,
sets in the search region, multiple candidate regions, each in a size corresponding to the region of interest,
obtains a norm between a pixel value of the region of interest and a pixel value of the candidate region, for each of the multiple candidate regions, thereby obtaining a norm distribution within the search region, and
generates an image assuming a value representing a state of the norm distribution as the pixel value of the region of interest that is associated with the search region.

2. The ultrasound imaging apparatus according to claim 1, wherein,
the norm is p-norm that is expressed by the following formula (1);
$$p \text{-} Norm = {( {\sum }_{i , j} {\vert {p}_{m} ( {i}_{0} , {j}_{0} ) - {p}_{m + Δ} ( i , j ) \vert}^{p} )}^{1 / p} \qquad ( 1 )$$
and Pₘ(i₀, j₀) represents the pixel value of the pixel at a position (i₀, j₀) within the region of interest, Pₘ+Δ(i, j) represents the pixel value of the pixel at the position (i, j) within the candidate region, and p represents a real number being predetermined.

3. The ultrasound imaging apparatus according to claim 2, wherein,
the p represents a real number larger than 1.

4. The ultrasound imaging apparatus according to claim 1, wherein,
the value representing the state of the norm distribution indicates statistics of the distribution.

5. The ultrasound imaging apparatus according to claim 4, wherein,
the statistics indicate a rate of divergence that is defined by a difference between a minimum norm value and an average value of the norm values, in the norm distribution within the search region.

6. The ultrasound imaging apparatus according to claim 4, wherein,
the statistics indicate a coefficient of variation that is obtained by dividing a standard deviation of the norm values by an average value, in the norm distribution within the search region.

7. The ultrasound imaging apparatus according to claim 1, wherein,
the processor
obtains a first direction and a second direction, out of multiple directions centering on a specific region being set within the search region;
the first direction in which the average of the norm values becomes a minimum in the candidate regions located along the direction, and the second direction passing through the specific region and being orthogonal to the first direction, and
uses a value of ratio or a value of difference between the average of the norm values in the candidate regions along the first direction and the average of the norm values in the candidate regions along the second direction, as the value representing the state of the norm distribution as to the region of the interest that is associated with the search region.

8. The ultrasound imaging apparatus according to claim 1, wherein,
the processor
sets a central pixel at the center of the candidate region,
sets multiple directions centering on the central pixel,
obtains a norm value between a pixel value of the central pixel and pixel values of multiple pixels located along each of the directions,
obtains a norm value average by dividing the norm value being obtained by the number of pixels along the directions, and
uses as the value of the central pixel of the candidate region, a ratio value or a difference value between the norm value average of a first direction in which the norm value average becomes minimum, and the norm value average obtained as to the central pixel and multiple pixels located along a second direction being orthogonal to the first direction passing through the central pixel.

9. The ultrasound imaging apparatus according to claim 7, wherein,
the processor applies enhancement to the norm distribution within the search region in advance, using the Laplacian filter, and the value of ratio or the value of difference is obtained as to the distribution after the enhancement.

10. The ultrasound imaging apparatus according to claim 8, wherein,
the processor applies enhancement to the pixel values within the candidate region in advance, using the Laplacian filter, and the value of ratio or the value of difference is obtained as to the pixel values after the enhancement.

11. The ultrasound imaging apparatus according to claim 1, wherein,
the processor generates a matrix representing the norm distribution within the search region, applies an eigenvalue decomposition process to the matrix to obtain an eigenvalue, and uses this eigenvalue as the value representing the state of the norm distribution as to the region of interest that is associated with the search region.

12. The ultrasound imaging apparatus according to claim 1, wherein,
the processor selects as a destination of the region of interest, the candidate region in which the norm value becomes minimum in the search region, and obtains a motion vector that connects a position of the region of interest and a position of the candidate region being selected, and generates the motion vector for each of the multiple regions of interest, thereby generating the motion vector field, and
the processor further obtains as a boundary norm value, a total sum of a squared value of derivative of y direction with respect to x component and a squared value of derivative of x direction with respect to y component, as to each of multiple specific regions set in the motion vector field, and generates an image assuming the boundary norm value as the pixel value of the specific region.

13. The ultrasound imaging apparatus according to claim 1, wherein,
the processor sets the multiple regions of interest in a manner partially overlapping,
and upon calculating the norm as to one of the regions of interest, the processor stores a value obtained with regard to the overlapping region in a lookup table in a storage region,
and the processor reads the value from the lookup table and uses the value, upon calculating the norm as to one of the other regions of interest.

14. The ultrasound imaging apparatus according to claim 1, wherein,
the processor sets the multiple candidate regions in a manner partially overlapping, and upon calculating the norm as to one of the candidate regions, the processor stores a value obtained with regard to the overlapping region in a lookup table in a storage region, and the processor reads the value from the lookup table and uses the value, upon calculating the norm as to one of the other candidate regions.

15. The ultrasound imaging apparatus according to claim 1, wherein,
the processor generates multiple frames of images on a time-series basis, the image being generated assuming the value representing the state of the norm distribution as the pixel value, and the processor calculates an amount of information entropy for each of the frames, and
when the amount of information entropy is equal to or larger than a predetermined threshold, the processor displays the frame.

16. The ultrasound imaging apparatus according to claim 1, wherein,
the processor generate an extraction image that is obtained by extracting pixels each having a value representing the norm distribution, the value being equal to or larger than a predetermined value, and the processor displays the extraction image in a superimposed manner on a B-mode image.

17. The ultrasound imaging apparatus according to claim 16, wherein,
the processor generates a histogram as to the value representing the state of the norm distribution and the frequency thereof, with regard to the image that is generated assuming the value representing the state of the norm distribution as the pixel value, and
the processor searches the histogram for a bell-shaped distribution, and uses a minimum value of the bell-shaped distribution as the predetermined value.

18. An ultrasound imaging method comprising,
transmitting an ultrasound wave to an object, and processing a received signal obtained by receiving the ultrasound wave coming from the object to generate images of two or more frames,
selecting two frames from the images,
setting multiple regions of interest in one of the frames,
setting in the other frame, search regions each wider than the region of interest, respectively for the multiple regions of interest, setting in the search region, multiple candidate regions, each in a size corresponding to the region of interest,
obtaining a norm between a pixel value of the region of interest and a pixel value of the candidate region, for each of the multiple candidate regions, thereby obtaining a norm distribution within the search region, and
generating an image assuming a value representing a state of the norm distribution as the pixel value of the region of interest that is associated with the search region.

19. An ultrasound imaging program for causing a computer to execute,
a first step of selecting two frames from two or more frames of ultrasound images,
a second step of setting multiple regions of interest in one of the frames,
a third step of setting in the other frame, search regions each wider than the region of interest, respectively for the multiple regions of interest, and setting in the search region, multiple candidate regions each in a size corresponding to the region of interest,
a fourth step of obtaining a norm between a pixel value of the region of interest and a pixel value of the candidate region, for each of the multiple candidate regions, thereby obtaining a norm distribution within the search region, and
a fifth step of generating an image assuming a value representing a state of the norm distribution as the pixel value of the region of interest that is associated with the search region.

20. An ultrasound imaging apparatus comprising,
a transmitter configured to transmit an ultrasound wave to an object,
a receiver configured to receive the ultrasound wave coming from the object, and
a processor configured to process a received signal in the receiver and generate images of two or more frames, wherein,
the processor
sets multiple regions of interest in a received signal distribution corresponding to one frame, out of the received signals associated with the images of two or more frames being received,
sets in the received signal distribution corresponding to the other frame, search regions each wider than the region of interest, respectively for the multiple regions of interest,
sets in the search region multiple candidate regions, each in a size corresponding to the region of interest,
obtains a norm between an amplitude distribution or a phase distribution of the region of interest and the amplitude distribution or the phase distribution of the candidate region, for each of the multiple candidate regions, thereby obtaining a norm distribution within the search region, and
generates an image assuming a value representing a state of the norm distribution as the pixel value of the region of interest that is associated with the search region.
